(19) United States
(12) Patent Application Publication
(10) Pub. No.: US 2024/0197852 A1
(43) Pub. Date: Jun. 20, 2024

(54) PSORALEN-INACTIVATED NEISSERIA GONORRHOEAE VACCINES AND METHODS THEREOF

(71) Applicants: Wake Forest University Health Sciences, Winston-Salem, NC (US); UNITED STATES AS REPRESENTED BY SECRETARY OF NAVY, Silver Spring, MD (US)

(72) Inventors: Leigh Ann Sanders, Winston-Salem, NC (US); John W. Sanders, Winston-Salem, NC (US); Marlena Westcott, Winston-Salem, NC (US); Kevin Porter, Silver Spring, MD (US)

(21) Appl. No.: 18/556,443

(22) PCT Filed: Apr. 22, 2022
(86) PCT No.: PCT/US22/25968

§ 371 Date: Oct. 20, 2023

Related U.S. Application Data
(60) Provisional application No. 63/201,326, filed on Apr. 23, 2021.

(51) Int. Cl. A61K 39/095; A61K 39/00; A61K 39/02; A61K 39/108; A61K 39/112; A61K 41/17; A61P 31/04; A61P 37/04

(52) U.S. Cl. CPC A61K 39/095; A61K 39/0258; A61K 39/0283; A61K 39/105; A61K 41/17; A61P 31/04; A61P 37/04; A61K 2039/521; A61K 2039/575

(57) ABSTRACT

The present invention relates to the fields of sexually transmitted disease and immunology. More specifically, but not exclusively, the invention provides a composition and methods to prepare and administer a vaccine for Neisseria gonorrhoede and/or other gram-negative bacteria such as by using psoralen-inactivated bacteria.

### PRIORITY STATEMENT

This application claims the benefit, under 35 U.S.C. § 119(e), of U.S. Provisional Application Ser. No. 63/201,326, filed Apr. 23, 2021, the entire contents of which are incorporated by reference herein.

### STATEMENT OF GOVERNMENT SUPPORT

This invention was made with government support under grant number 982210 awarded by the Department of Defense. The government has certain rights in the invention.

### FIELD OF INVENTION

The present invention relates to the fields of sexually transmitted disease and immunology. More specifically, but not exclusively, the invention provides a composition and methods to prepare and administer a vaccine for Neisseria gonorrhoeae and/or other gram-negative bacteria such as by using psoralen- and UV-inactivated bacteria.

### BACKGROUND OF INVENTION

Neisseria gonorrhoeae is the etiological agent of gonorrhea, the second most frequently reported sexually transmitted infection in the world [1]. According to the World Health Organization (WHO), there were 78 million new cases of gonococcal infection in people aged 15-49 worldwide during 2012 [2].

Gonorrhea typically infects the genital tract in men and women. Symptoms include urethritis, pain and/or a burning sensation when urinating, urethral discharge and painful testicles for men. Women can experience an increase in vaginal discharge, vaginal bleeding, pain and/or a burning sensation when urinating.

Gonorrhea rates are increasing in at risk populations globally and antimicrobial resistance among gonococcal (GC) isolates is growing concurrently. [3] The U.S. Centers for Disease Control (CDC) identified multi-drug resistant gonococcus (MDR GC) as an “urgent” hazard-level threat. [4] There are also potentially significant sequelae to GC infection such as Pelvic Inflammatory Disease (PID) which can lead to infertility or ectopic pregnancy. [5] Control of the spread of infection is complicated by the high rate of asymptomatic infection, occurring in up to 30% of men and >50% of women. [6] There is a pressing need for an effective GC vaccine to respond to this threat.

Unfortunately, it is not yet clear that an effective GC vaccine can be developed. Reinfection after uncomplicated genital infection occurs and natural infection appears to only induce a weak and transient mucosal immune response. [7] Furthermore, GC infection appears to stimulate immune suppression or diversion. [8] Correlates of protective immunity, if they exist, still need to be clarified. [7]

However, a recent analysis of the impact of protein-based meningococcal group B vaccine (MeNZB, Bexsero, GSK) on GC infection rates in New Zealand has provided renewed hope and enthusiasm for a vaccine option.[9] A retrospective case-control study at sexual health clinics of patients aged 15-30 years eligible to receive MeNZB was performed. Cases were confirmed by laboratory isolation or detection of only Neisseria gonorrhoeae and controls were individuals only positive for Chlamydia (CT). There were 14,730 total cases and controls: 1,241 GC infections, 12,487 CT infections, and 1,002 with co-infections. Vaccinated individuals were significantly less likely to be cases than controls (511 [41%] vs 6424 [51]; adjusted OR 0.69 [95% CI 0.61-0.79]; p<0.0001). Estimated vaccine effectiveness against gonorrhea was 31% (95% CI 21-39). [9] While a 30% efficacy might seem too low to be worthwhile, epidemiological modeling of the impact of vaccination in a heterosexual population with standardized risk of acquisition suggests that a reduction in prevalence approaching 40% could be achieved with a vaccine of only 20% efficacy.

### SUMMARY OF THE INVENTION

The described invention relates to a Neisseria gonorrhoeae bacterial vaccines prepared by a variety of methods, including but not limited to inactivating Neisseria gonorrhoeae through exposure to psoralen and/or by exposure to ultraviolet light. In some embodiments, after initial exposure, bacteria are exposed a second time to psoralen followed by ultraviolet light. Without wishing to be bound to theory, this inactivation method inactivates the cell without altering epitopes that may be important for generating protective immune responses. This method of preparation may be used on other bacteria including, but not limited to Shigella and Campylobacter jejuni. The vaccine is prepared with the inactivated Neisseria gonorrhoeae with or without an adjuvant.

Accordingly, one aspect of the present invention provides an immunogenic composition comprising a crosslinked inactivated bacterium, wherein the bacterium has been crosslinked and inactivated by contact with psoralen or a psoralen derivative and exposure to ultraviolet (UV) irradiation.

In some embodiments, the bacterium is a gram negative bacterium, e.g., Neisseria gonorrhoeae, Escherichia coli (e.g., enterotoxigenic E. coli, uropathogenic E. coli, and the like), Shigella, and/or Campylobacter jejuni.

In some embodiments, the psoralen or psoralen derivative is selected from the group consisting of 8-Methoxypsoralen (8-MOP), 4,5′,8-Trimethylpsoralen (TMP), psoralen derivative 4-aminomethyl-4,5′, 8-trimethylpsoralen (AMT) and a combination thereof.

Another aspect of the present invention provides a method of making an immunogenic composition (e.g., the immunogenic composition of the present invention), comprising: a) contacting a live bacterium to psoralen or psoralen derivative in a medium to produce a bacterium/psoralen mixture, and b) exposing the mixture of step (a) to an intensity of ultraviolet (UV) radiation for a time period sufficiently long to crosslink the live bacterium and render the live bacterium inactive.

Also provided herein is an immunogenic composition produced by the methods of the present invention.

Another aspect of the present invention provides a method of producing an immune response to a bacterium in a subject, comprising administering to the subject an effective amount of the immunogenic composition of the present invention, thereby producing an immune response to a bacterium in the subject.

Another aspect of the present invention provides a method of treating a bacterial infection in a subject in need thereof, comprising administering to the subject an effective amount of the immunogenic composition of the present invention, thereby treating a bacterial infection in the subject.

Another aspect of the present invention provides a method of preventing or reducing the risk of a disease or disorder associated with or caused by a bacterial infection in a subject, comprising administering to the subject an effective amount of the immunogenic composition of the present invention, thereby preventing or reducing the risk of a disease or disorder associated with or caused by a bacterial infection in the subject.

Another aspect of the present invention provides a method of protecting a subject from the effects of a bacterial infection, comprising administering to the subject an effective amount of the immunogenic composition of the present invention, thereby protecting the subject from the effects of a bacterial infection.

Another aspect of the present invention provides a method of preventing or reducing the risk of a gonococcal infection or disorder associated with or caused by a gonococcal infection in a subject, comprising administering to the subject an effective amount of the immunogenic composition of the present invention, thereby preventing or reducing the risk of a disease or disorder associated with or caused by a bacterial infection in the subject.

Another aspect of the present invention provides a method of protecting a subject from the effects of a gonococcal infection, comprising administering to the subject an effective amount of the immunogenic composition of the present invention, thereby protecting the subject from the effects of a bacterial infection.

In some embodiments, the immunogenic composition may be administered prior to the subject exhibiting symptoms of the infection, such as wherein the immunogenic composition is administered prophylactically (e.g., to non-infected subjects, e.g., as a prophylactic vaccine).

In some embodiments, the immunogenic composition may be administered for one, two, three, or more repetitions (e.g., boosting doses).

Also provided herein is a vaccine comprising the immunogenic composition of the present invention.

Also provide herein is a vaccine against Neisseria gonorrhoeae comprising a psoralen-inactivated Neisseria gonorrhoeae.

Another aspect of the present invention provides a method for inactivating a live bacterium for vaccine use comprising: a) exposing said bacterium to an inactivating psoralen or psoralen derivative, b) exposing said bacterium to a preselected intensity of an ultraviolet radiation, and c) repeating exposure to said inactivating psoralen or psoralen derivative followed by exposure to said ultraviolet radiation for a time period sufficiently long to render the bacteria inactive.

In some embodiments, the bacterium may be Neisseria gonorrhoeae, Shigella, Campylobacter jejuni, Enterotoxigenic Escherichia coli, and/or uropathogenic Escherichia coli.

Another aspect of the present invention provides a method for making a vaccine of the present invention, wherein said psoralen-inactivated Neisseria gonorrhoeae is added to a pharmaceutically acceptable carrier in sufficient concentration to create an immune response.

Another aspect of the present invention provides a method for immunizing a subject against bacterial infection, comprising administering a vaccine of the present invention.

### BRIEF DESCRIPTION OF THE DRAWINGS

FIG. 1 depicts OD₄₅₀ values from pooled sera. The psoralen-killed vaccines generated significantly greater antibody responses than did the formalin-killed vaccines, as shown by OD₄₅₀ vs dilution of pooled sera. Serum IgM+G+A ELISA, Day 14 post-boost. Anti-Neisseria antibodies (IgM+G+A) in pooled sera collected at 14 days after a boost with each of the indicated vaccines. Data points represent triplicate determinations from a representative experiment.

FIG. 2 depicts titers from the pools, calculated based on serial dilutions of serum performed in 2 independent ELISA tests. Anti-Neisseria serum IgM+G+A ELISA. Serum antibody levels in pooled sample (n=7-8 mice/group) collected at 14 days post-boost. Titer is defined as serum dilution that yielded an OD₄₅₀ value equivalent to that of a control anti-Neisseria monoclonal antibody diluted 1:85,000. The mean s.d. from 2 experiments is shown. Expressing the data as titers, Formalin-killed was approximately 5,000, and increased to approximately 35,000 in the presence of dmLT. The titer for the psoralen-killed vaccine was approximately 60,000 and was increased to approximately 200,000 in the presence of dmLT.

FIG. 3 depicts antibody titers from individual mice and the geometric mean titer is indicated. Anti-Neisseria serum antibody titers (IgM+G+A) 14 days post-boost. Line indicates geometric mean titer (GMT). Significance determined by 2-tailed, unpaired Student's test. ** p<0.01; *** p<0.001; **** p<0.0001.

### DETAILED DESCRIPTION OF THE INVENTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of +10%, +5%, +1%, +0.5%, or even+0.1% of the specified value as well as the specified value. For example, “about X” where X is the measurable value, is meant to include X as well as variations of +10%, +5%, +1%, +0.5%, or even+0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof. As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, “one or more” means one, two, three, four, five, six, seven, eight, nine, ten, 11, 12, 13, 14, 15, 16, 17, 18, 19, 20, etc.

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

As used herein, by “isolate” or “purify” (or grammatical equivalents) a compound (e.g., an antibody), it is meant that the compound is at least partially separated from at least some of the other components in the starting material.

The term “endogenous” refers to a component naturally found in an environment, i.e., a gene, nucleic acid, miRNA, protein (e.g., antibody), cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

The terms “immunogen” and “antigen” are used interchangeably herein and mean any compound (including polypeptides) to which a cellular and/or humoral immune response can be directed. A molecule capable of antibody and/or immune response stimulation may be referred to as antigenic/immunogenic, and can be said to have the ability of antigenicity/immunogenicity. The binding site for an antibody within an antigen and/or immunogen may be referred to as an epitope (e.g., an antigenic epitope). The term “vaccine antigen” as used herein refers to such an antigen/immunogen as used as a vaccine, e.g., a prophylactic, preventative, and/or therapeutic vaccine.

The term “administering” or “administration” of a composition of the present invention to a subject includes any route of introducing or delivering to a subject a compound to perform its intended function (e.g., for use as a therapeutic). Administration includes self-administration and the administration by another.

“Pharmaceutically acceptable” as used herein means that the compound, carrier, or composition is suitable for administration to a subject to achieve a treatment described herein, without unduly deleterious side effects in light of the severity of the disease and necessity of the treatment.

As used herein, the term “therapeutically effective amount” refers to an amount of a product of the present invention (e.g., including but not limited to a compound, composition, adjuvant, carrier and/or an immunogenic composition of the present invention) that elicits a therapeutically useful response in a subject. Those skilled in the art will appreciate that the therapeutic effects need not be complete or curative, as long as some benefit is provided to the subject.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with bacterial infection (e.g., gonococcal infection) is achieved and/or there is a delay in the progression of the symptom. In some embodiments, the severity of a symptom associated with bacterial infection (e.g., gonococcal infection) may be reduced in a subject compared to the severity of the symptom in the absence of a method of the present invention.

A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a composition of the present invention.

The terms “prevent,” “preventing” and “prevention” (and grammatical variations thereof) refer to avoidance, reduction and/or delay of the onset of a symptom associated with bacterial infection (e.g., gonococcal infection) and/or a reduction in the severity of the onset of symptom associated with bacterial infection (e.g., gonococcal infection) relative to what would occur in the absence of a method of the present invention. The prevention can be complete, e.g., the total absence of the symptom. The prevention can also be partial, such that the occurrence of the symptom in the subject and/or the severity of onset is less than what would occur in the absence of a method of the present invention.

A “prevention effective” amount as used herein is an amount that is sufficient to prevent (as defined herein) a symptom associated with bacterial infection (e.g., gonococcal infection) in a subject. Those skilled in the art will appreciate that the level of prevention need not be complete, as long as some benefit is provided to the subject. In some embodiments, a prevention effective amount may be achieved by administering a composition of the present invention.

The present invention finds use in both veterinary and medical applications. Subjects suitable to be treated with a method of the present invention include, but are not limited to, mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of all genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult.

In some embodiments, the subject is “in need of” or “in need thereof” a method of the present invention, for example, the subject has findings typically associated with bacterial infection (e.g., gonococcal infection). In some embodiments, a subject “in need of” or “in need thereof” a method of the present invention a subject previously having, having, suspected of having, or at risk of bacterial infection (e.g., gonococcal infection), including, for example, a non-infected subject as a candidate for a prophylactic vaccine.

A method of the present invention may also be carried out on animal subjects, particularly mammalian subjects such as mice, rats, dogs, cats, livestock and horses for veterinary purposes, and/or for drug screening and drug development purposes.

A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art.

The term “antibody” or “antibodies” as used herein refers to all types of immunoglobulins, including IgG, IgM, IgA, IgD, and IgE. The antibody can be monoclonal or polyclonal and can be of any species of origin, including, for example, mouse, rat, rabbit, horse, goat, sheep or human, or can be a chimeric or humanized antibody. See, e.g., Walker et al., Molec. Immunol. 26:403-11 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980. The antibody can further be a single chain antibody or bispecific antibody. The antibody can also be humanized for administration to a human subject.

Non-limiting examples of an antibody or fragment thereof of the present invention include a monoclonal antibody or fragment thereof, a chimeric antibody or fragment thereof, a CDR-grafted antibody or fragment thereof, a humanized antibody or fragment thereof, an Fc, a Fab, a Fab′, a F(ab′)2, a Fv, a disulfide linked Fv, a single chain antibody (scFv), a single domain antibody (dAb), a diabody, a multispecific antibody (e.g., a bispecific antibody) or fragment thereof, an anti-idiotypic antibody or fragment thereof, a bifunctional hybrid antibody or fragment thereof, a functionally active epitope-binding antibody fragment, an affibody, a nanobody, and any combination thereof. Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab)2 fragments. Alternatively, Fab expression libraries can be constructed to allow rapid and easy identification of monoclonal Fab fragments with the desired specificity (Huse et al., (1989) Science 254:1275-1281).

As used herein, the term “gonococcal infection” refers to any infection by the organism Neisseria gonorrhoeae, also referred to as gonococcus (singular) or gonococci (plural) or abbreviated as GC. Gonococcal infections of the present invention include but are not limited to sexually transmitted gonorrhea (e.g., primary genitourinary gonorrhea), disseminated gonococcemia, gonococcal septicemia (e.g., septic arthritis), and/or perinatal infection such as but not limited to neonatal conjunctivitis or gonococcal ophthalmia neonatorum. Non-limiting examples of N. gonorrhoeae include GC strain FA1090 (ATCC #700825), GC CDC reference strain ATCC #49226, and the 2016 W.H.O. GC reference strains described in Unemo et al. 2016 J. Antimicrob. Chemother. 71(11):3096-3108, incorporated herein by reference.

### Compositions

One aspect of the present invention provides an immunogenic composition comprising a crosslinked inactivated bacterium, wherein the bacterium has been crosslinked and inactivated by contact with psoralen or a psoralen derivative and exposure to ultraviolet (UV) irradiation.

The bacterium may be any bacterium wherein treatment with psoralen or a psoralen derivative and exposure to ultraviolet irradiation leads to replicative inactivation (“killed”), including but not limited to a state of being killed but metabolically active (“KBMA”). Without wishing to be bound to theory, it is believed that psoralen plus ultraviolet radiation inhibits replication but does not affect protein structure or prevent translation, protein epitopes remain conserved and psoralen inactivated organisms (e.g., bacterial cells and/or viral particles) remain in a KBMA state for at least a period of time.

In some embodiments, the bacterium is a gram-negative bacterium. In some embodiments, the bacterium is Neisseria gonorrhoeae, Escherichia coli (e.g., enterotoxigenic E. coli, uropathogenic E. coli, and the like), Shigella, and/or Campylobacter jejuni.

The psoralen or psoralen derivative of the invention may be any natural and/or synthetic psoralen-related compound known or later discovered or generated. Psoralen is a naturally occurring molecule comprising a planar, tricyclic structure of a furan ring bound to a coumarin moiety. Psoralen and psoralen derivatives are members of a family of organic compounds known as furanocoumarins, are mutagenic and intercalate with nucleic acids. As used herein, the term “psoralen derivative” refers to any natural, modified, and/or fully synthetic compound derived from and/or comprising the base structure of psoralen. While not wishing to be bound to theory, psoralen and psoralen derivatives may become covalently attached to thymine upon exposure to UV irradiation, leading to nucleic acid crosslinking. Non-limiting examples of a psoralen or a psoralen derivative of this invention include 8-Methoxypsoralen (8-MOP), 4,5′,8-Trimethylpsoralen (TMP), psoralen derivative 4-aminomethyl-4,5′, 8-trimethylpsoralen (AMT) and a combination thereof. Additional non-limiting examples of psoralens and psoralen derivatives contemplated in this invention include those described in Buhimschi et al. 2020 Photochem. Photobiol. 96(5): 1014-1031, incorporated herein by reference.

The UV radiation contemplated in the present invention may be UV radiation of any wavelength, including but not limited to about 400 nm to about 100 nm, e.g., about 400, 375, 350, 325, 300, 275, 250, 225, 200, 175, 150, 125, or 100 nm or any value or range therein. In some embodiments, the UV radiation contemplated for the compositions and methods of the present invention may comprise UVA irradiation (e.g., about 400 nm to about 315 nm). In some embodiments, the UV radiation may comprise about 365 nm.

In some embodiments, the inactivated crosslinked bacterium may be in a composition at a concentration of about 5×10⁶ colony forming unit (CFU) equivalents to about 5×10⁸ CFU equivalents, e.g., about 1, 2, 3, 4, 5, 6, 7, 8, 9×10⁶ CFU equivalents, about 1, 2, 3, 4, 5, 6, 7, 8, 9×10⁷ CFU equivalents, or about 1, 2, 3, 4, 5, 6, 7, 8, 9 10⁸ CFU equivalents, or any value or range therein. For example, in some embodiments, the inactivated crosslinked bacterium may be in a composition at a concentration of about 5×10⁶ CFU equivalents, about 5×10⁷ CFU equivalents, or about 1×10⁶ CFU equivalents to about 9×10⁸ CFU equivalents, about 5×10⁶ CFU equivalents to about 5×10⁸ CFU equivalents, or about 1×10⁷ CFU equivalents to about 1×10⁸ CFU equivalents. As used herein, the term “colony forming unit” or “CFU” refers to a quantification of the number of bacterial cells in a sample which are viable (e.g., can multiply to form colonies). CFU quantification is standard in the art and methods of measuring CFU will be readily apparent to the skilled artisan. Similarly, the term “CFU equivalents” as used herein refers to a concentration of inactivated crosslinked bacterium within a composition from which an amount (e.g. a sample) is ex vivo and/or in vitro quantified to comprise a particular amount of colony forming units (CFU) as measured, for example, by standard microbiological plating methods as would be known to the skilled artisan.

In some embodiments, the immunogenic composition of the present invention may further comprise an adjuvant, optionally in a vaccine diluent. As used herein, “suitable adjuvant” describes an adjuvant capable of being combined with an immunogen, immunogenic composition, pharmaceutical composition, or vaccine of this invention to further enhance an immune response without deleterious effect on the subject or the cell of the subject.

The adjuvant of the present invention may comprise any standard adjuvant known in the field relevant to vaccine preparations, as would be readily known to the skilled artisan upon review of the disclosures herein. The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24(6): 766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

Adjuvants can be combined, either with the compositions of this invention or with other vaccine compositions that can be used in combination with the compositions of this invention.

In some embodiments, the adjuvant may be aluminum hydroxide, Aso4 and/or dmLT.

In some embodiments, a composition (e.g., an immunogenic composition, pharmaceutical composition and/or vaccine) of the present invention may comprise an adjuvant at a concentration of about 0.25 μg/ml to about 250 μg/ml, e.g., about 0.25, 0.50, 0.75, 1, 1.5, 2, 3, 4, 5, 6, 7, 8, 9, 10, 20, 30, 40, 50, 60, 70, 80, 90, 100, 125, 150, 175, 200, 225, or 250 μg/ml or any value or range therein. For example, in some embodiments, the composition may comprise the adjuvant at a concentration of about 0.25 μg/ml, about 1 μg/ml, about 2.5 μg/ml, about 20 μg/ml, about 40 μg/ml, about 200 μg/ml, or about 250 μg/ml. In some embodiments, the composition may comprise an adjuvant at a concentration of about 0.25 μg/ml to about 200 μg/ml, about 2.5 μg/ml to about 40 μg/ml, or about 1 μg/ml to about 100 μg/ml.

In some embodiments, an immunogenic composition of the present invention may further comprise a pharmaceutically acceptable carrier. By “pharmaceutically acceptable” it is meant a material that is not toxic or otherwise undesirable, i.e., the material may be administered to a subject without causing any undesirable biological effects. In some embodiments, the present invention provides a pharmaceutical composition comprising a crosslinked and inactivated bacterium and/or immunogenic composition comprising the same, a pharmaceutically acceptable carrier, and, optionally, other medicinal agents, therapeutic agents, pharmaceutical agents, stabilizing agents, buffers, carriers, adjuvants, diluents, etc., which can be included in the composition singly or in any combination and/or ratio. Such compositions, especially vaccines, are typically prepared as injectables, either as liquid solutions or suspensions. Solid forms suitable for solution in, or suspension in, liquid prior to injection may also be prepared. Lyophilized preparations are also suitable. For injection, the carrier will typically be a liquid. For other methods of administration, the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

Active immunogenic ingredients may be mixed with excipients and/or carriers that are pharmaceutically acceptable and/or compatible with the active ingredient(s). Suitable excipients include but are not limited to sterile water, saline, dextrose, glycerol, ethanol, or the like and combinations thereof, as well as stabilizers, e.g., HSA or other suitable proteins and reducing sugars. In addition, if desired, vaccines or immunogenic compositions of the present invention may contain minor amounts of auxiliary substances such as wetting and/or emulsifying agents, pH buffering agents, and/or adjuvants that enhance the effectiveness of the vaccine or immunogenic composition.

In some embodiments, a pharmaceutical or immunogenic composition of the present invention may further comprise additional agents, such as, but not limited to, additional antigen as part of a cocktail in a vaccine, e.g., a multi-component vaccine wherein the vaccine may additionally include nucleic acids, bacterial peptides, bacterial cells, virus, viral peptides, etc.

In some embodiments, the bacterium of the present invention may be inactivated by UV irradiation via exposure to said UV irradiation for about 1 to about 30 minutes, e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or any value or range therein. For example, in some embodiments the bacterium is inactivated by UV irradiation via exposure to said UV irradiation for about 2 to about 3 minutes, about 2 to about 30 minutes, about 1 to about 5 minutes, or about 60 seconds, about 90 seconds, about 120 seconds (2 minutes), about 3 minutes, about 5 minutes, or about 10 minutes or more.

In some embodiments, the bacterium of the present invention may be inactivated by UV irradiation via exposure to said UV irradiation at a preselected intensity. By “preselected intensity,” reference is made to an intensity of UV irradiation wherein either single and/or repeated exposure to said intensity for a certain time period and/or repetitions sufficiently renders the bacteria inactive, including but limited to killed but metabolically active (KBMA). In some embodiments, the preselected intensity may be an intensity of about 0.01 Joule/cm² to about 100 Joule/cm², e.g., about 0.01, 0.05, 0.10, 0.15, 0.20, 0.30, 0.40, 0.50, 0.60, 0.70, 0.80, 0.90, 1, 1.5, 2, 3, 4, 5, 6, 7, 8, 9, 10, 20, 30, 40, 50, 60, 70, 80, 90, 91, 92, 93, 94, 95, 96, 97, 98, 99, or 100 Joule/cm² or any value or range therein. Accordingly, in some embodiments, the bacterium of the present invention may be inactivated by UV irradiation via exposure to said UV irradiation at an intensity of about 0.01 Joule/cm² to about 100 Joule/cm², e.g., about 0.01, 0.05, 0.10, 0.15, 0.20, 0.30, 0.40, 0.50, 0.60, 0.70, 0.80, 0.90, 1, 1.5, 2, 3, 4, 5, 6, 7, 8, 9, 10, 20, 30, 40, 50, 60, 70, 80, 90, 91, 92, 93, 94, 95, 96, 97, 98, 99, or 100 Joule/cm² or any value or range therein. For example, in some embodiments the bacterium of the present invention may be inactivated by UV irradiation via exposure at an intensity of about 0.01 Joule/cm², about 1 Joule/cm², about 0.5 Joule/cm², about 4 Joule/cm², about 10 Joule/cm², about 50 Joule/cm², about 100 Joule/cm², or about 0.01 Joule/cm² to about 100 Joule/cm², about 0.5 Joule/cm² to about 50 Joule/cm², about 1 Joule/cm² to about 10 Joule/cm², or about 0.1 Joule/cm² to about 20 Joule/cm².

Also provided herein is a vaccine comprising an immunogen and/or immunogenic composition of the present invention. For example, in some embodiments, a vaccine against Neisseria gonorrhoeae comprising a psoralen-inactivated Neisseria gonorrhoeae (e.g., a psoralen-inactivated Neisseria gonorrhoeae immunogen) is provided. Such vaccine and/or psoralen-inactivated immunogen may be generated via any of the methods as described above or below.

For example, in some embodiments, said Neisseria gonorrhoeae immunogen is inactivated by: a) exposing said Neisseria gonorrhoeae to an inactivating psoralen or psoralen derivative, b) exposing said Neisseria gonorrhoeae to a preselected intensity of an ultraviolet radiation, and c) repeating exposure to said inactivating psoralen or psoralen derivative followed by exposure to said ultraviolet radiation for a time period sufficiently long to render the bacteria inactive. In some embodiments, UV exposure of step (b) occurs in the presence of the psoralen or psoralen derivative exposed in step (a).

In some embodiments, said psoralen or psoralen derivative of the inactivated immunogen is selected from the group consisting of, 8-Methoxypsoralen (8-MOP), 4,5′,8-Trimethylpsoralen (TMP), psoralen derivative 4-aminomethyl-4,5′, 8-trimethylpsoralen (AMT) and a combination thereof.

In some embodiments, said psoralen exposed to the inactivated immunogen is added to a medium (e.g., a culture medium, e.g., GCP and/or LB broth) at a concentration of about 0.01 to about 600 μg/ml (e.g., about 0.01, 0.05, 0.10, 0.15, 0.20, 0.25, 0.50, 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 435, 450, 475, 500, 525, 550, 575, 580, 590, 591, 592, 593, 594, 595, 596, 597, 598, 599, or 600 μg/ml or any value or range therein. In some embodiments, said concentration of said psoralen is about 20 μg/ml.

In some embodiments, the ultraviolet radiation exposure of the inactivated immunogen is about 2 to about 30 minutes, e.g., about 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or any value or range therein. In some embodiments, said ultraviolet radiation exposure is about 2 to about 3 minutes.

In some embodiments, the intensity of ultraviolet radiation is about 0.01 Joule/cm² to about 100 Joule/cm², e.g., about 0.01, 0.05, 0.10, 0.15, 0.20, 0.30, 0.40, 0.50, 0.60, 0.70, 0.80, 0.90, 1, 1.5, 2, 3, 4, 5, 6, 7, 8, 9, 10, 20, 30, 40, 50, 60, 70, 80, 90, 91, 92, 93, 94, 95, 96, 97, 98, 99, or 100 Joule/cm² or any value or range therein. In some embodiments, the intensity of ultraviolet radiation is about 1 Joule/cm².

In some embodiments, said psoralen-inactivated Neisseria gonorrhoeae is added to a pharmaceutically acceptable carrier in sufficient concentration to create an immune response upon administration to a subject.

In some embodiments, said psoralen-inactivated Neisseria gonorrhoeae concentration is a concentration of about 5×10⁶ CFU equivalents to about 5×10⁸ CFU equivalents, e.g., about 1, 2, 3, 4, 5, 6, 7, 8, 9×10⁶ CFU equivalents, about 1, 2, 3, 4, 5, 6, 7, 8, 9×10⁷ CFU equivalents, or about 1, 2, 3, 4, 5, 6, 7, 8, 9 10⁸ CFU equivalents, or any value or range therein. For example, in some embodiments, said inactivated Neisseria gonorrhoeae bacterium is diluted to a concentration of about 5 ×10⁷ CFU equivalents.

In some embodiments, an adjuvant is added to said psoralen-inactivated Neisseria gonorrhoeae immunogen and/or vaccine. In some embodiments, said adjuvant is aluminum hydroxide, Aso4 and/or dmLT. In some embodiments, said dmLT adjuvant is added at about 0.25 μg/ml to about 250 μg/ml, e.g., about 0.25, 0.50, 0.75, 1, 1.5, 2, 3, 4, 5, 6, 7, 8, 9, 10, 20, 30, 40, 50, 60, 70, 80, 90, 100, 125, 150, 175, 200, 225, or 250 μg/ml or any value or range therein. In some embodiments, said adjuvant is added at about 2.5 μg/ml.

### Methods of Making

Also provided herein are methods of making any of the immunogens and/or compositions (e.g., immunogenic compositions, pharmaceutical compositions, and/or vaccines) of the present invention. The compositions of this invention are intended for use as prophylactic agents, therapeutic agents and immunological reagents, for example, as antigens, immunogens, and/or vaccines. The compositions described herein can be formulated for use as reagents (e.g., to produce antibodies) and/or for administration in a pharmaceutical carrier in accordance with known techniques. See, e.g., Remington, The Science and Practice of Pharmacy (latest edition).

Accordingly, one aspect of the present invention provides a method of making an immunogenic composition (e.g., an immunogenic composition as described above) comprising: a) contacting a live bacterium to psoralen or psoralen derivative in a medium to produce a bacterium/psoralen mixture, and b) exposing the mixture of step (a) to an intensity of UV radiation for a time period sufficiently long to crosslink the live bacterium and render the live bacterium inactive. Bacterial inactivation may be measured by any known and standard method in the art, as will be readily apparent to the skilled artisan upon review of the disclosures herein, such as but not limited to as measured by post-inactivation bacterial plating (e.g., colony forming units (CFU) quantification).

Media contemplated for the methods of the present invention include any medium which allows for growth of a relevant bacterium, with or without additional supplementation as needed (e.g., a culture medium). Many known and standard culture media will be known to and readily applicable by the skilled artisan. For example, in some embodiments, the medium may be an inactivation medium or a cell culture medium in which the bacterium is grown. In some embodiments, the medium may be a standard gonococcal medium, i.e. “GC” broth (also referred to as “GCP”). In some embodiments, the medium may be LB broth or the like.

In some embodiments, the live bacterium may comprise a population of live bacteria, e.g., a population of a gram-negative bacterium such as but not limited to Neisseria gonorrhoeae, Escherichia coli (e.g., enterotoxigenic E. coli, uropathogenic E. coli, and the like), Shigella, and/or Campylobacter jejuni.

In some embodiments, the step (b) exposing the mixture of step (a) to an intensity of UV radiation for a time period sufficiently long to render the live bacterium inactive comprises repeating steps (a) and/or (b) iteratively until complete inactivation is achieved. As used herein, the term “complete inactivation” refers to the condition wherein no new growth of the previously live bacterium can be measured, for example as quantified by bacterial CFU quantification following steps (a) and (b)). In some embodiments, the previously live bacterium may be completely inactivated, while remaining killed but metabolically active (KBMA). In some embodiments, the previously live bacterium may be completely inactivated, including metabolically inactive. For example, in some embodiments, the step (b) exposing the mixture of step (a) to an intensity of UV radiation for a time period sufficiently long to render the live bacterium inactive may comprise repeating steps (a) and/or (b) for one or more, two or more, three or more, or four or more iterations. In some embodiments, the step (b) exposing the mixture of step (a) to an intensity of UV radiation for a time period sufficiently long to render the live bacterium inactive may comprise repeating steps (a) and/or (b) for one more iteration, for a total of two rounds of step (a) and step (b).

In some embodiments, following an initial round of step (a) and (b), the mixture may be incubated for a time period between iterations of additional step(s) (a) and/or step(s) (b), e.g., may be incubated for a time period following the immediately prior step (a) prior to initiating the next step (b) and/or may be incubated for a time period following the immediately prior step (b) prior to initiating the next step (a). In some embodiments, the mixture may be incubated for a time period between step (a) and step (b). In some embodiments, the mixture may be incubated for a time period of at least 1, 2, 3, 4, 5, 6, 7, 8, 9, 10, 11, 12, 13, 14, 15, 16, 17, 18, 19, 20, 21, 22, 23, 24, 25, 26, 27, 8, 29, 30, 35, 40, 45, 50, 55, 60 or more minutes, e.g., about 1, 2, 3, 4, 5, 6 or more hours, or any value or range therein, between step (a) and step (b), and/or between iterations of additional step(s) (a) and step(s) (b). In some embodiments, the psoralen or psoralen derivative (e.g., 8-Methoxypsoralen (8-MOP), 4,5′,8-Trimethylpsoralen (TMP), psoralen derivative 4-aminomethyl-4,5′, 8-trimethylpsoralen (AMT) and a combination thereof) may be added to the medium at a concentration of about 0.01 to about 600 μg/ml, e.g., about 0.01, 0.05, 0.10, 0.15, 0.20, 0.25, 0.50, 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 435, 450, 475, 500, 525, 550, 575, 580, 590, 591, 592, 593, 594, 595, 596, 597, 598, 599, or 600 μg/ml or any value or range therein. In some embodiments, the concentration of said psoralen or psoralen derivative may be about 20 μg/ml. In some embodiments, the concentration of said psoralen or psoralen derivative may be about 40 μg/ml.

The exposure time of the UV radiation may be any singular time and/or cumulative time of repeated exposures which is sufficiently long to render the bacterium inactive and/or KBMA. For example, in some embodiments, UV radiation exposure is about 2 to about 30 minutes, e.g., about 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or any value or range therein. In some embodiments, the ultraviolet radiation exposure is about 2 to about 3 minutes.

The intensity of the UV radiation may be any intensity which sufficiently renders the bacterium inactive and/or KBMA upon any singular UV exposure and/or cumulative repeated exposures, e.g., a preselected intensity. For example, in some embodiments, the intensity of UV radiation contemplated for the methods of the present invention may be about 0.01 Joule/cm² to about 100 Joule/cm² (e.g., about 0.01, 0.05, 0.10, 0.15, 0.20, 0.30, 0.40, 0.50, 0.60, 0.70, 0.80, 0.90, 1, 1.5, 2, 3, 4, 5, 6, 7, 8, 9, 10, 20, 30, 40, 50, 60, 70, 80, 90, 91, 92, 93, 94, 95, 96, 97, 98, 99, or 100 Joule/cm² or any value or range therein. For example, in some embodiments the intensity of UV radiation contemplated for the methods of the present invention may be about 0.01 Joule/cm², about 1 Joule/cm², about 0.5 Joule/cm², about 4 Joule/cm², about 10 Joule/cm², about 50 Joule/cm², about 100 Joule/cm², or about 0.01 Joule/cm² to about 100 Joule/cm², about 0.5 Joule/cm² to about 50 Joule/cm², about 1 Joule/cm² to about 10 Joule/cm², or about 0.1 Joule/cm² to about 20 Joule/cm².

Also provided herein is a method for inactivating a live bacterium for vaccine use comprising: a) exposing said bacterium to an inactivating psoralen or psoralen derivative, b) exposing said bacterium to a preselected intensity of an ultraviolet radiation, and c) repeating exposure to said inactivating psoralen or psoralen derivative followed by exposure to said ultraviolet radiation for a time period sufficiently long to render the bacteria inactive.

In some embodiments, said inactivating psoralen is introduced to said medium in about 1 to about 4 additions.

In some embodiments, said medium is an inactivation medium or a cell culture medium in which the bacterium is grown.

In some embodiments, the produced psoralen-inactivated Neisseria gonorrhoeae is added to a pharmaceutically acceptable carrier in sufficient concentration to create an immune response.

In some embodiments, the produced psoralen-inactivated Neisseria gonorrhoeae may be prepared to a concentration of about 5×10⁶ CFU equivalents to about 5×10⁸ CFU equivalents, e.g., about 5×10⁷ CFU equivalents.

In some embodiments, an adjuvant may be added.

Adjuvant, UV conditions (intensity, time period and repletion of exposures), and psoralen or psoralen derivative conditions applicable to the method may be any of those as described above.

Also provided herein is any composition (e.g., immunogen, immunogenic composition, pharmaceutical composition, and/or vaccine) produced by the methods disclosed herein.

### Methods of Using

Also provided herein are methods of using the compositions (e.g., immunogen, immunogenic composition, pharmaceutical composition, and/or vaccine) disclosed herein and/or produced by the methods disclosed herein.

Accordingly, another aspect of the present invention provides a method of producing an immune response to a bacterium in a subject, comprising administering to the subject an effective amount of an immunogenic composition of the present invention, thereby producing an immune response to a bacterium in the subject.

Another aspect of the present invention provides a method of treating a bacterial infection in a subject in need thereof, comprising administering to the subject an effective amount of the immunogenic composition of the present invention, thereby treating a bacterial infection in the subject.

Another aspect of the present invention provides a method of preventing or reducing the risk of a disease or disorder associated with or caused by a bacterial infection in a subject, comprising administering to the subject an effective amount of the immunogenic composition of the present invention, thereby preventing or reducing the risk of a disease or disorder associated with or caused by a bacterial infection in the subject.

Another aspect of the present invention provides a method of protecting a subject from the effects of a bacterial infection, comprising administering to the subject an effective amount of the immunogenic composition of the present invention, thereby protecting the subject from the effects of a bacterial infection.

Also provided herein is a method for immunizing a subject against bacterial infection, comprising administering a composition of the present invention (e.g., the immunogen, immunogenic composition, pharmaceutical composition, and/or vaccine of the present invention).

In some embodiments, the subject may comprise a subject previously having, having, suspected of having, or at risk of infection by a bacterium (e.g., a gram-negative bacterium). For example, in some embodiments, the subject may comprise a subject previously having, having, suspected of having, or at risk of infection by Neisseria gonorrhoeae, Escherichia coli (e.g., enterotoxigenic E. coli, uropathogenic E. coli, and the like), Shigella, and/or Campylobacter jejuni. In some embodiments, the subject comprises a subject previously having, having, suspected of having, or at risk of infection by Neisseria gonorrhoeae.

In some embodiments, the subject is a mammal, e.g., a human. In some embodiments, the mammalian subject is a human, e.g., a human patient.

Accordingly, also provided herein is a method of preventing or reducing the risk of a gonococcal infection or disorder associated with or caused by a gonococcal infection (e.g., a Neisseria gonorrhoeae infection) in a subject, comprising administering to the subject an effective amount of the immunogenic composition of the present invention, thereby preventing or reducing the risk of a disease or disorder associated with or caused by a gonococcal infection in the subject.

Also provided herein is a method of protecting a subject from the effects of a gonococcal infection (e.g., a Neisseria gonorrhoeae infection), comprising administering to the subject an effective amount of the immunogenic composition of the present invention, thereby protecting the subject from the effects of a bacterial infection.

In some embodiments, the immunogenic composition may be administered prior to the subject exhibiting symptoms of the infection e.g. wherein the immunogenic composition is administered prophylactically, e.g., to non-infected subjects, e.g., as a prophylactic vaccine.

In some embodiments, the immunogenic composition may be administered in a “prime-boost” strategy, e.g., wherein the immunogenic composition is administered for one, two, three, or more repetitions (e.g., boosting doses).

As used herein, the terms “prime boost immunization,” “prime boost administration,” or “prime and booster” refer to an administration (e.g., immunization) regimen that comprises administering to a subject a primary/initial (priming) administration (e.g., of one or more immunogen, immunogenic composition, pharmaceutical composition, and/or vaccine of the present invention) and at least one secondary (boosting) administration. In some embodiments, the priming administration and the at least one boosting administration may comprise the same composition, administered in multiple (one or more) repetitions. In some embodiments, the priming administration and the at least one boosting administration may comprise different types of compositions, such as different types of immunogens, immunogenic compositions, pharmaceutical compositions, and/or vaccines of the present invention.

As used herein, the terms “prime immunization,” “priming immunization,” “primary immunization” or “prime” refer to primary antigen stimulation by using an immunogen, immunogenic composition, pharmaceutical composition, and/or vaccine of the present invention according to the instant invention.

The term “boost immunization,” “boosting immunization,” “secondary immunization”, “boost,” or “booster” refers to additional administration (e.g., immunization) of a composition of the present invention administered to a subject after a primary administration. In some embodiments, the boost immunization may be administered at a dose higher than, lower than, and/or equal to the dose administered as a primary immunization, e.g., when the boost immunization is administered alone without priming.

The prime and boost vaccine compositions may be administered via the same route or they may be administered via different routes. The boost vaccine composition may be administered one or several times at the same or different dosages. It is within the ability of one of skill in the art to optimize prime-boost combinations, including optimization of the timing and dose of vaccine administration.

In some embodiments, the one or more repetitions (boosting doses) may be administered at least one, two, three, four, or more weeks after the initial administering step (e.g., the priming administration). In some embodiments, the one or more repetitions (boosting doses) may be administered three weeks (21 days) after the initial administering step (e.g., the priming administration).

The compositions of the present invention (e.g., immunogen, immunogenic composition, pharmaceutical composition, and/or vaccine) may be administered in any frequency, amount, and/or route as needed to elicit an effective prophylactic and/or therapeutic effect in a subject (e.g., in a subject in need thereof) as described herein. The most suitable route in any given case will depend, as is well known in the art, on such factors as the species, age, gender and overall condition of the subject, the nature and severity of the condition being treated and/or on the nature of the particular composition (i.e., dosage, formulation) that is being administered. Non-limiting examples of suitable means of administration include intraperitoneally, intramuscularly, intranasally, intravenously, intradermally, intrarectally and/or subcutaneously. The compositions herein may be administered via a skin scarification method, and/or transdermally via a patch or liquid. The compositions can be delivered subdermally in the form of a biodegradable material that releases the compositions over a period of time. As further non-limiting examples, the route of administration can be by inhalation (e.g., oral and/or nasal inhalation), oral, buccal (e.g., sublingual), rectal, vaginal, topical (including administration to the airways), intraocular, by parenteral (e.g., intramuscular [e.g., administration to skeletal muscle], intravenous, intra-arterial, intraperitoneal and the like), subcutaneous (including administration into the footpad), intrapleural, intracerebral, intrathecal, intraventricular, intra-aural, intra-ocular (e.g., intra-vitreous, sub-retinal, anterior chamber) and peri-ocular (e.g., sub-Tenon's region) routes or any combination thereof. In some embodiments, the immunogenic composition is administered in a route selected from the group consisting of oral, transdermal, epidermal, transcutaneous, mucosa, intravaginal, and intrarectal.

In some embodiments, the immunogenic composition may be administered with or without an adjuvant.

Also provided herein is a method for immunizing a subject against bacterial infection, comprising administering a composition of the present invention (e.g., the immunogen, immunogenic composition, pharmaceutical composition, and/or vaccine of the present invention).

Formulations for use in the methods disclosed herein include injectables, which can be prepared in conventional forms, either as liquid solutions or suspensions, solid forms suitable for solution or suspension in liquid prior to injection, or as emulsions. Pharmaceutical compositions suitable for oral administration can be presented in discrete units, such as capsules, cachets, lozenges, or tablets, each containing a predetermined amount of the composition of this invention; as a powder or granules; as a solution or a suspension in an aqueous or non-aqueous liquid; or as an oil-in-water or water-in-oil emulsion. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions suitable for parenteral administration can comprise sterile aqueous and non-aqueous injection solutions of the composition of this invention, which preparations are optionally isotonic with the blood of the intended recipient. These preparations can contain antioxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit/dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces) and transdermal administration. The compositions herein may also be administered via a skin scarification method, or transdermally via a patch or liquid. The compositions may be delivered subdermally in the form of a biodegradable material that releases the compositions over a period of time. The most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for rectal administration can be presented as unit dose suppositories. These can be prepared by admixing the composition with one or more conventional solid carriers, such as for example, cocoa butter, and then shaping the resulting mixture.

Pharmaceutical compositions of this invention suitable for topical application to the skin can take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharm. Res. 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

The delivery methods disclosed herein may be administered to the lungs of a subject by any suitable means, for example, by administering an aerosol suspension of respirable particles comprised of the compositions, which the subject inhales. The respirable particles may be liquid or solid. Aerosols of liquid particles comprising the compositions of the invention may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compositions of the invention may likewise be produced with any solid particulate medicament aerosol generator, by techniques known in the pharmaceutical art.

The compositions of this invention can be optimized and combined with other vaccination regimens to provide the broadest (i.e., covering all aspects of the immune response, including those features described hereinabove) cellular and humoral responses possible.

The present invention may be as defined in any one of the following numbered paragraphs.

1. An immunogenic composition comprising a crosslinked inactivated bacterium, wherein the bacterium is crosslinked and inactivated by contact with psoralen or a psoralen derivative and exposure to ultraviolet (UV) irradiation.

2. The immunogenic composition of paragraph 1, wherein the bacterium is a gram-negative bacterium.

3. The immunogenic composition of paragraph 1 or 2, wherein the bacterium is Neisseria gonorrhoeae, Escherichia coli (e.g., enterotoxigenic E. coli, uropathogenic E. coli, and the like), Shigella, and/or Campylobacter jejuni.

4. The immunogenic composition of any one of paragraphs 1-3, wherein said psoralen or psoralen derivative is selected from the group consisting of 8-Methoxypsoralen (8-MOP), 4,5′,8-Trimethylpsoralen (TMP), psoralen derivative 4-aminomethyl-4,5′, 8-trimethylpsoralen (AMT) and a combination thereof.

5. The immunogenic composition of any one of paragraphs 1-4, wherein the UV irradiation comprises UVA irradiation.

6. The immunogenic composition of any one of paragraphs 1-5, comprising the inactivated crosslinked bacterium in a concentration of about 5×10⁶ CFU equivalents to about 5×10⁸ CFU equivalents (e.g., about 1, 2, 3, 4, 5, 6, 7, 8, 9×10⁶ CFU equivalents, about 1, 2, 3, 4, 5, 6, 7, 8, 9×10⁷ CFU equivalents, or about 1, 2, 3, 4, 5, 6, 7, 8, 9 10⁸ CFU equivalents, or any value or range therein).

7. The immunogenic composition of paragraph 6, comprising the inactivated crosslinked bacterium in a concentration of about 5×10⁷ CFU equivalents.

8. The immunogenic composition of any one of paragraphs 1-7, further comprising an adjuvant.

9. The immunogenic composition of paragraph 8, wherein the adjuvant is aluminum hydroxide, Aso4 and/or dmLT.

10. The immunogenic composition of paragraph 9, wherein the composition comprises the adjuvant at a concentration of about 0.25 μg/ml to about 250 μg/ml (e.g., about 0.25, 0.50, 0.75, 1, 1.5, 2, 3, 4, 5, 6, 7, 8, 9, 10, 20, 30, 40, 50, 60, 70, 80, 90, 100, 125, 150, 175, 200, 225, or 250 μg/ml or any value or range therein).

11. The immunogenic composition of paragraph 10, wherein the composition comprises the adjuvant at a concentration of about 2.5 μg/ml.

12. The immunogenic composition of any one of paragraphs 1-11, further comprising a pharmaceutically acceptable carrier.

13. The immunogenic composition of any one of paragraphs 1-12, wherein the bacterium is inactivated by UV irradiation via exposure to said UV irradiation for about 2 to about 30 minutes (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or any value or range therein).

14. The immunogenic composition of paragraph 13, wherein the bacterium is inactivated by UV irradiation via exposure to said UV irradiation for about 2 to about 3 minutes.

15. The immunogenic composition of any one of paragraphs 1-14, wherein the bacterium is inactivated by UV irradiation via exposure to said UV irradiation at an intensity of about 0.01 Joule/cm² to about 100 Joule/cm² (e.g., about 0.01, 0.05, 0.10, 0.15, 0.20, 0.30, 0.40, 0.50, 0.60, 0.70, 0.80, 0.90, 1, 1.5, 2, 3, 4, 5, 6, 7, 8, 9, 10, 20, 30, 40, 50, 60, 70, 80, 90, 91, 92, 93, 94, 95, 96, 97, 98, 99, or 100 100 Joule/cm² or any value or range therein).

16. The immunogenic composition of paragraph 15, wherein the bacterium is inactivated by UV irradiation via exposure to said UV irradiation at an intensity of about 1 Joule/cm².

17. A method of making an immunogenic composition (e.g., the immunogenic composition of any one of paragraphs 1-16), comprising: a) contacting a live bacterium (e.g., a gram-negative bacterium) to psoralen or psoralen derivative in a medium (e.g., a culture medium, e.g., GC broth (“GCP”), LB broth, etc.) to produce a bacterium/psoralen mixture, and b) exposing the mixture of step (a) to an intensity of ultraviolet (UV) radiation (e.g., UVA radiation) for a time period sufficiently long to crosslink the live bacterium and render the live bacterium inactive (e.g., as measured by post-inactivation bacterial plating).

18. The method of paragraph 17, wherein the live bacterium comprises a population of live bacteria (e.g., a population of a gram-negative bacterium such as but not limited to Neisseria gonorrhoeae, Escherichia coli (e.g., enterotoxigenic E. coli, uropathogenic E. coli, and the like), Shigella, and/or Campylobacter jejuni).

19. The method of paragraph 17 or 18, wherein the step (b) exposing the mixture of step (a) to an intensity of ultraviolet (UV) radiation for a time period sufficiently long to render the live bacterium inactive comprises repeating steps (a) and/or (b) iteratively until complete inactivation (e.g., repeating steps (a) and/or (b) for one or more, two or more, three or more, or four or more iterations).

20. The method of paragraph 19, wherein the steps (a) and/or (b) are repeated for one or more iterations (e.g., at least two total rounds of step (a) and/or step (b).

21. The method of paragraph 19 or 20, wherein the mixture is incubated for a time period (e.g., for a time period of at least 1, 2, 3, 4, 5, 6, 7, 8, 9, 10, 11, 12, 13, 14, 15, 16, 17, 18, 19, 20, 21, 22, 23, 24, 25, 26, 27, 8, 29, 30, 35, 40, 45, 50, 55, 60 or more minutes, e.g., about 1, 2, 3, 4, 5, 6 or more hours, or any value or range therein) between iterations of additional step (a) contacting with psoralen or a psoralen derivative and/or step (b) exposing the mixture of step (a) (e.g., the immediately prior step (a)) to the ultraviolet (UV) radiation.

22. The method of any one of paragraphs 17-21, wherein said psoralen or psoralen derivative is selected from the group consisting of, 8-Methoxypsoralen (8-MOP), 4,5′,8-Trimethylpsoralen (TMP), psoralen derivative 4-aminomethyl-4,5′, 8-trimethylpsoralen (AMT) and a combination thereof.

23. The method of any one of paragraphs 17-22, wherein said psoralen is added to the medium at a concentration of about 0.01 to about 600 μg/ml (e.g., about 0.01, 0.05, 0.10, 0.15, 0.20, 0.25, 0.50, 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 435, 450, 475, 500, 525, 550, 575, 580, 590, 591, 592, 593, 594, 595, 596, 597, 598, 599, or 600 μg/ml or any value or range therein).

24. The method of paragraph 23, wherein said concentration of said psoralen is about 20 g/ml.

25. The method of any one of paragraphs 17-24, wherein the UV radiation exposure is about 2 to about 30 minutes (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or any value or range therein).

26. The method of paragraph 25, wherein said ultraviolet radiation exposure is about 2 to about 3 minutes.

27. The method of any one of paragraphs 27-26, wherein the intensity of UV radiation is about 0.01 Joule/cm² to about 100 Joule/cm² (e.g., about 0.01, 0.05, 0.10, 0.15, 0.20, 0.30, 0.40, 0.50, 0.60, 0.70, 0.80, 0.90, 1, 1.5, 2, 3, 4, 5, 6, 7, 8, 9, 10, 20, 30, 40, 50, 60, 70, 80, 90, 91, 92, 93, 94, 95, 96, 97, 98, 99, or 100 100 Joule/cm² or any value or range therein).

28. The method of paragraph 27, wherein the intensity of ultraviolet radiation is about 1 Joule/cm².

29. An immunogenic composition produced by the methods of any one of paragraphs 17-28.

30. A method of producing an immune response to a bacterium (e.g., a gram-negative bacterium) in a subject, comprising administering to the subject an effective amount of the immunogenic composition of any one of paragraphs 1-16 or 29, thereby producing an immune response to a bacterium in the subject.

31. A method of treating a bacterial infection (e.g., a gram-negative bacterial infection) in a subject in need thereof, comprising administering to the subject an effective amount of the immunogenic composition of any one of paragraphs 1-16 or 29, thereby treating a bacterial infection in the subject.

32. A method of preventing or reducing the risk of a disease or disorder associated with or caused by a bacterial infection (e.g., a gram-negative bacterial infection) in a subject, comprising administering to the subject an effective amount of the immunogenic composition of any one of paragraphs 1-16 or 29, thereby treating a bacterial infection in the subject, thereby preventing or reducing the risk of a disease or disorder associated with or caused by a bacterial infection in the subject.

33. A method of protecting a subject from the effects of a bacterial infection (e.g., a gram-negative bacterial infection), comprising administering to the subject an effective amount of the immunogenic composition of any one of paragraphs 1-16 or 29, thereby protecting the subject from the effects of a bacterial infection.

34. The method of any one of paragraphs 30-33, wherein the subject comprises a subject previously having, having, suspected of having, or at risk of infection by a bacterium (e.g., a gram-negative bacterium).

35. The method of any one of paragraphs 30-34, wherein the bacterium is Neisseria gonorrhoeae, Escherichia coli (e.g., enterotoxigenic E. coli, uropathogenic E. coli, and the like), Shigella, and/or Campylobacter jejuni.

36. A method of preventing or reducing the risk of a gonococcal infection or disorder associated with or caused by a gonococcal infection (e.g., a Neisseria gonorrhoeae infection) in a subject, comprising administering to the subject an effective amount of the immunogenic composition of any one of paragraphs 1-16 or 29, thereby treating a bacterial infection in the subject, thereby preventing or reducing the risk of a disease or disorder associated with or caused by a bacterial infection in the subject.

37. A method of protecting a subject from the effects of a gonococcal infection (e.g., a Neisseria gonorrhoeae infection), comprising administering to the subject an effective amount of the immunogenic composition of any one of paragraphs 1-16 or 29, thereby protecting the subject from the effects of a bacterial infection.

38. The method of paragraph 36 or 37, wherein the subject comprises a subject previously having, having, suspected of having, or at risk of infection by Neisseria gonorrhoeae.

39. The method of any one of paragraphs 31-38, wherein the immunogenic composition is administered prior to the subject exhibiting symptoms of the infection (e.g. wherein the immunogenic composition is administered prophylactically, e.g., to non-infected subjects, e.g., as a prophylactic vaccine).

40. The method of any one of paragraphs 30-39, wherein the immunogenic composition is administered for one, two, three, or more repetitions (e.g., boosting doses).

41. The method of paragraph 40, wherein the one or more repetitions (boosting doses) is administered at least one, two, three, four, or more weeks after the initial administering step (e.g., the priming administration).

42. The method of paragraph 41, wherein the one or more repetitions (boosting doses) is administered three weeks (21 days) after the initial administering step (e.g., the priming administration).

43. The method of any one of paragraphs 30-42, wherein the subject is a mammal (e.g., a human).

44. The method of paragraph 43, wherein the mammal is a human (e.g., a human patient).

45. The method of any one of paragraphs 30-44, wherein the immunogenic composition is administered in a route selected from the group consisting or oral, transdermal, epidermal, transcutaneous, mucosa, intravaginal, and intrarectal.

46. The method of any one of paragraphs 30-45, wherein the immunogenic composition is administered with or without an adjuvant.

46b. A vaccine comprising the immunogenic composition of any one of paragraphs 1-16 or 29.

47. A vaccine against Neisseria gonorrhoeae comprising a psoralen-inactivated Neisseria gonorrhoeae.

48. The vaccine of paragraph 47, wherein said Neisseria gonorrhoeae is inactivated by:

a) exposing said Neisseria gonorrhoeae to an inactivating psoralen or psoralen derivative, b) exposing said Neisseria gonorrhoeae to a preselected intensity of an ultraviolet radiation, and c) repeating exposure to said inactivating psoralen or psoralen derivative followed by exposure to said ultraviolet radiation for a time period sufficiently long to render the bacteria inactive.

49. The vaccine of paragraph 48, wherein said psoralen or psoralen derivative is selected from the group consisting of, 8-Methoxypsoralen (8-MOP), 4,5′,8-Trimethylpsoralen (TMP), psoralen derivative 4-aminomethyl-4,5′, 8-trimethylpsoralen (AMT) and a combination thereof.

50. The vaccine of paragraph 48, wherein said psoralen is added to a medium at a concentration of about 0.01 to about 600 μg/ml (e.g., about 0.01, 0.05, 0.10, 0.15, 0.20, 0.25, 0.50, 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 435, 450, 475, 500, 525, 550, 575, 580, 590, 591, 592, 593, 594, 595, 596, 597, 598, 599, or 600 μg/ml or any value or range therein).

51. The vaccine of paragraph 48, wherein said concentration of said psoralen is about 20 μg/ml.

52. The vaccine of paragraph 48, wherein said ultraviolet radiation exposure is about 2 to about 30 minutes (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or any value or range therein).

53. The vaccine of paragraph 48, wherein said ultraviolet radiation exposure is about 2 to about 3 minutes.

54. The vaccine of paragraph 48, wherein the intensity of ultraviolet radiation is about 0.01 Joule/cm² to about 100 Joule/cm² (e.g., about 0.01, 0.05, 0.10, 0.15, 0.20, 0.30, 0.40, 0.50, 0.60, 0.70, 0.80, 0.90, 1, 1.5, 2, 3, 4, 5, 6, 7, 8, 9, 10, 20, 30, 40, 50, 60, 70, 80, 90, 91, 92, 93, 94, 95, 96, 97, 98, 99, or 100 100 Joule/cm² or any value or range therein).

55. The vaccine of paragraph 48, wherein the intensity of ultraviolet radiation is about 1 Joule/cm².

56. The vaccine of paragraph 47, wherein said psoralen-inactivated Neisseria gonorrhoeae is added to a pharmaceutically acceptable carrier in sufficient concentration to create an immune response.

57. The vaccine of paragraph 47, wherein said psoralen-inactivated Neisseria gonorrhoeae concentration is a concentration of about 5×10⁶ CFU equivalents to about 5×10⁸ CFU equivalents (e.g., about 1, 2, 3, 4, 5, 6, 7, 8, 9×10⁶ CFU equivalents, about 1, 2, 3, 4, 5, 6, 7, 8, 9×10⁷ CFU equivalents, or about 1, 2, 3, 4, 5, 6, 7, 8, 9 10⁸ CFU equivalents, or any value or range therein).

58. The vaccine of paragraph 47, wherein said inactivated Neisseria gonorrhoeae bacterium is diluted to a concentration of about 5×10⁷ CFU equivalents.

59. The vaccine of paragraph 47, wherein an adjuvant is added.

60. The vaccine of paragraph 59, wherein said adjuvant is aluminum hydroxide, Aso4 and/or dmLT.

61. The vaccine of paragraph 60, wherein said dmLT adjuvant is added at about 0.25 μg/ml to about 250 μg/ml (e.g., about 0.25, 0.50, 0.75, 1, 1.5, 2, 3, 4, 5, 6, 7, 8, 9, 10, 20, 30, 40, 50, 60, 70, 80, 90, 100, 125, 150, 175, 200, 225, or 250 μg/ml or any value or range therein).

62. The vaccine of paragraph 61, wherein said dmLT adjuvant is added at about 2.5 μg/ml.

63. A method for inactivating a live bacterium for vaccine use comprising: a) exposing said bacterium to an inactivating psoralen or psoralen derivative, b) exposing said bacterium to a preselected intensity of an ultraviolet radiation, and c) repeating exposure to said inactivating psoralen or psoralen derivative followed by exposure to said ultraviolet radiation for a time period sufficiently long to render the bacteria inactive.

64. The method of paragraph 63, wherein said bacterium is Neisseria gonorrhoeae.

65. The method of paragraph 63, wherein said bacterium is Shigella. 66. The method of paragraph 63, wherein said bacterium is Campylobacter jejuni.

67. The method of paragraph 63, wherein said bacterium is Enterotoxigenic Escherichia coli.

68. The method of paragraph 63, wherein said bacterium is Uropathogenic Escherichia coli.

69. The method of paragraph 63, wherein said inactivating psoralen is added to a medium containing said live bacterium.

70. The method of paragraph 69, wherein said inactivating psoralen is introduced to said medium in about 1 to about 4 additions.

71. The method of paragraph 69, wherein said medium is an inactivation medium or a cell culture medium in which the bacterium is grown.

72. The method of paragraph 63, wherein said psoralen or psoralen derivative is selected from the group consisting of 8-Methoxypsoralen (8-MOP), 4,5′,8-Trimethylpsoralen (TMP), psoralen derivative 4-aminomethyl-4,5′, 8-trimethylpsoralen (AMT) and a combination thereof.

73. The method of paragraph 72, wherein said psoralen is added to the medium at a concentration of about 0.01 to about 600 μg/ml.

74. The method of paragraph 72, wherein the concentration of said psoralen is about 20 μg/ml.

75. The method of paragraph 63, wherein said ultraviolet radiation exposure is about 2 to about 30 minutes.

76. The method as in paragraph 63, wherein said ultraviolet radiation exposure is about 2 to about 3 minutes.

77. The method of paragraph 63, wherein the intensity of ultraviolet radiation is about 0.01 Joule/cm² to about 100 Joule/cm².

78. The method of paragraph 63, wherein the intensity of ultraviolet radiation is about 1 Joule/cm².

79. A method for making the vaccine of paragraph 47, wherein said psoralen-inactivated Neisseria gonorrhoeae is added to a pharmaceutically acceptable carrier in sufficient concentration to create an immune response.

80. The method of paragraph 79, wherein said psoralen-inactivated Neisseria gonorrhoeae concentration is a concentration of about 5×10⁶ CFU equivalents to about 5×10⁸ CFU equivalents

81. The method of paragraph 79, wherein said psoralen-inactivated Neisseria gonorrhoeae concentration is a concentration of about 5×10⁷ CFU equivalents.

82. The method of paragraph 79, wherein an adjuvant is added.

83. The method of paragraph 82, wherein said adjuvant is aluminum hydroxide, Aso4 or dmLT.

84. The method of paragraph 83, wherein said dmLT adjuvant is added at about 0.25 μg/ml to about 250 μg/ml.

85. The method of paragraph 83, wherein said dmLT adjuvant is added at about 2.5 μg/ml.

86. A method for immunizing a subject against bacterial infection, comprising administering the vaccine of paragraph 47.

87. The method of paragraph 86, wherein said vaccine is administered in a route selected from the group consisting: oral, transdermal, epidermal, transcutaneous, mucosa, intravaginal, and intrarectal.

88. The method of paragraph 86, wherein said vaccine is administered with or without an adjuvant.

89. The method of paragraph 88, wherein said adjuvant is aluminum hydroxide, Aso4 and/or dmLT.

90. The method of paragraph 86, further comprising one or more boosting dose of said vaccine.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1

According to the present invention, vaccines useful for the inoculation of against infection by Neisseria gonorrhoeae are provided. In some embodiments, the vaccines may be prepared by inactivating live Neisseria gonorrhoeae in a medium containing inactivating psoralen and then exposing the mixture to ultraviolet radiation. After initial exposure, a new portion of psoralen may be added and the mixture may again be exposed to ultraviolet radiation.

In some embodiments, vaccines may be prepared by combining the inactivated bacterium with a pharmaceutically acceptable carrier in sufficient concentration to create an immune response. In some embodiments, an adjuvant may be added to help increase the immune response.

Strains and Growth Conditions: GC strain FA1090 AATCC #700825 and E. coli strain H10407 ATCC #35401 were purchased through ATCC. FA1090 uvrB mutant strains 1-81-S2 (uvrB:kan #4 Opa+) and 1-81-S2 (uvrB:kan #9) were obtained from the Seifert laboratory. GC were grown at 37ºC with 5% CO2 on GC Chocolate II agar with hemoglobin and IsoVitalex purchased from BD BBL. Broth cultures were cultivated in GCP stock supplemented with 0.042% sodium bicarbonate and 0.01% Isovitalex (BD BBL). [13, 14] Frozen stocks of GC strains and of psoralen inactivated GC cells were kept in GCP broth and 25% glycerol E. coli strain H10407 was cultivated in LB at 37º C.

Chemicals: The psoralen derivative AMT (4-aminomethyl-4,5′, 8-trimethylpsoralen), 8-MOP (8-methoxypsoralen), and Formalin were purchased from Sigma.

Formalin Inactivation of GC: GC strain FA1090 was grown overnight on GC agar plates. Overnight growth (between 16 and 18 hours) was resuspended in 5 mls of GCP and OD₆₀₀ was adjusted to 1.0. Cells were diluted 2:25 in 5 mls GCP supplemented with sodium bicarbonate and Isovitalex and grown at 37º C in the shaker for 6 hours (when the OD₆₀₀ is between 0.5 and 0.8). Growth was stopped by the addition of up to 10% formalin and left to mix with gentle shaking for two hours at room temperature. Samples were plated on GC agar to check for growth.

Psoralen Inactivation: GC strains were grown overnight on GC agar plates. Overnight growth (between 16 and 18 hours) was resuspended in 5 mls of GCP and OD₆₀₀ was adjusted to 1.0. Cells were diluted 2:25 in 5 mls GCP supplemented with sodium bicarbonate and Isovitalex and grown at 370C in the shaker for 7 hours at which time AMT was added and the cultures were incubated in the shaker for another hour. All bacterial cultures were then moved into 6 well cell culture plates and treated with 1 Joule/cm² UV light using an Analytik Jena Crosslinker with UVA light source (365 nm) (model #CL-1000L, Upland, CA). Cultures were returned to tubes and a second dose of AMT was added. Cultures were shaken for 1 hour at 370C and then crosslinked again at the same UV dose. Samples were washed in PBS and then plated on GC agar and incubated for two days at 370C and 5% CO2 to measure growth as an indication of the presence of residual CFU.

Early experiments on the effects of different doses of 8-MOP, AMT, and of UV on bacterial cells were done in E. coli strain H10407 due to its relative ease and speed of growth. H10407 was diluted 1:500 in LB and grown for 5 hours at 37ºC in a shaker. Psoralen inactivation was performed as described for GC. Bacterial cells were washed and inoculated into LB broth or plated onto LB agar to measure overnight growth as an indication of the presence of residual CFU.

Alternative Exposure Patterns to Psoralen and Ultraviolet (UVA) Light: In embodiments of the present invention, more than two additions of psoralen or psoralen derivatives are contemplated. In embodiments of the present invention, psoralen or psoralen derivatives are added at a low level, even including levels below 0.01 μg/ml, in more than four additions. Ultraviolet light can be added after each addition, in between any number of additions or after all additions have been complete.

In some embodiments, a continuous pump apparatus pumps a culture through a psoralen or psoralen addition chamber and then through ultraviolet light and then right back into an AMT addition chamber.

In some embodiments, cells are not instantly inactivated. For example, cells undergoing phase variation or responding to stress with different protein expression could potentially display a whole population of different antigens if they are inactivated over a longer period of time instead of all at once.

Formulation of Vaccine: Psoralen and formalin inactivated bacteria were washed three times in PBS and then adjusted to an OD₆₀₀ of 0.2 (approximately 1×10⁸ cells/ml). Vaccines were prepared to contain inactivated bacteria at 5×10⁷ CFU equivalents per ml+2.5 μg/ml dmLT in sterile PBS. Delivery of 0.1 ml per mouse yielded a dose of 5×10⁶ bacteria+0.25 μg dmLT.

Immunogenicity of Formalin Killed and Psoralen Killed FA1090: The experiments reported herein were conducted in compliance with the Animal Welfare Act and in accordance with the Principles set forth in Guide for the Care and Use of Laboratory Animals, National Research Council, National Academy Press, 1996. The study protocol was reviewed and approved by the Wake Forest University Animal Care and Use Committee (IACUC). Mice (6-8 weeks old) were purchased from Charles River. During the study mice were housed 4 per cage and fed on a standard diet of commercially produced mouse chow.

Groups of mice (7 or 8 per group) were immunized with either AMT inactivated FA1090, AMT inactivated FA1090 plus dmLT, formalin inactivated FA1090, or formalin inactivated FA 1090 plus dmLT. The vaccines were administered intramuscularly on days 0 and 21 and blood was collected from each group on days 0 (pre-bleed), 21, and 35 (14 days post-boost). For each time point some sera was pooled and tested for the presence of anti-Neisseria antibodies by whole cell Elisa.

Whole Cell ELISA: Bacteria were grown on GC plates for 16 hr. Cells were harvested and resuspended in PBS at an OD₆₀₀ of 0.2. Microtiter plates (Maxisorp, NUNC) were filled with 50 ul of the bacterial suspension and allowed to incubate overnight at 37 degrees Celsius. Wells were washed three times with PBS plus. 1% Tween 20 and then blocked for three hours with PBS+10% FCS. After blocking, cells were incubated with 100 ul of either a serially diluted mouse monoclonal anti-Neisseria IgG antibody (Invitrogen-need number) or sera from vaccinated mice for 1 hour at RT. The plates were washed and then incubated with 100 ul of HRP-conjugated goat anti mouse IgM+lgG+IgA secondary antibody (Millipore #AP501P) diluted 1:5000. After 45 minutes the plates were washed and 100 ul of TMB (3,3′, 5.5′-tetramethylbenzidine from Sigma) was added to each well. Plates were incubated at RT in the dark for 25 minutes and then 100 ul of a Stop solution (2M H2SO4) was added to each well. Absorption at 450 nm was measured using a BioTek EPOCH2NS spectrophotometer (BioTek Instruments, Inc, Highland Park, VT). The titer was defined as the dilution of mouse serum that yielded an OD₄₅₀ value equivalent to that of a positive control antibody (anti-Neisseria gonorrhoeae lgG antibody) using a pre-determined dilution from the linear range of the dose/response curve (1:85,000). The positive control antibody was included on each ELISA plate to normalize for plate to plate and day to day differences in OD₄₅₀ range. The serum titer was calculated by linear regression analysis of 3 to 4 Log10 transformed serum dilutions with OD₄₅₀ values that fell within the linear range of the dose/response curves using GraphPad Prism software.

Inactivation of E. coli with AMT+UVA light: Other labs had previously reported the use of the psoralen derivatives 8-MOP or S-59/psoralen [19-21] and UVA light to inactivate bacterial cultures containing DNA repair mutations. In order to determine if a bacterial culture not containing a DNA repair mutation with 8-MOP and UVA crosslinking could be inactivated, E. coli strain H10407 was used. Multiple separate cultures of this strain were treated with 50, 60, 70, or 80 μg/ml 8-MOP and treated with 1 Joule/cm² or 7 Joules/cm² UVA light and then diluted into LB broth and grown overnight. If even one cell in each culture survived the treatment with 8-MOP and UVA, it was expected to see growth in the overnight culture. Control cultures that received UVA but no 8-MOP, 8-MOP but no UVA, and no UVA or 8-MOP were also included. Heat killed cultures were diluted and grown overnight as a negative control. All cultures except the heat killed cultures grew up overnight, indicating that H10407 were not inactivated with these conditions.

We then used the psoralen derivative 4-aminomethyl-4,5′, 8-trimethylpsoralen (AMT) [22, 23]. Multiple separate cultures of the E. coli strain H10407 were treated with doses of psoralen derivative 4-aminomethyl-4,5′, 8-trimethylpsoralen (AMT) ranging from 0 to 60 μg/ml and treated with 1 Joule/cm², 7 Joules/cm², or 10 Joules/cm² UVA light and then diluted into LB broth and grown overnight. Overnight growth would indicate that there were surviving cells, while a lack of overnight growth would show that inactivation had occurred. Control cultures that received no treatment always grew up overnight as did cultures that only received AMT and no UVA or UVA treatment with no AMT. Heat killed cultures never grew up overnight. Cultures that received AMT and UVA light were sometimes completely inactivated and there was no growth in the overnight culture. (See Table 1). Cultures that had no growth were left to grow for 3 more days to make sure that no growth came up later.

Although none of the AMT doses completely inactivated E. coli every single time, the fact that some cultures were completely inactivated was somewhat unexpected. It is probable that a mutant lacking the ability to repair DNA crosslinks would be necessary for AMT treatment to be completely successful.

The desire was a method to inactivate the culture consistently with AMT. E. coli strain H10407 was treated with AMT and UVA as before, but then immediately retreated the same culture once again with AMT at the same dosage, allowing the culture to shake for 1 hour, and then crosslinking it a second time and plating the cells to see which cultures would still have surviving cells capable of growth. By plating the cells and comparing to the CFUs of an untreated culture we were able to determine a percentage of surviving cells. Multiple cultures were treated at every drug dose. (See Table 2)

The results showed that treating E. coli strain H10407 twice with 40 μg/ml AMT and 1 Joule/cm² of UVA eliminated all survivors. Thirty separate 1 ml cultures of H10407 were grown, treated with AMT at a dose of 40 μg/ml two times, and given 1 Joule/cm² of UVA two times and allowed to grow overnight on LB plates. None of these thirty cultures had a single surviving cell. LB plates were kept for multiple days to ensure there was no later growth. These results indicate that out of approximately 3.7×10¹⁰ cells (as determined by plating live cultures) treated with 40 μg/ml AMT and 1 Joule/cm² UV light two times, there were no cells still capable of growth.

Evaluating the use of a GC uvrB mutants: In order to examine the effect of a mutation in the uvrB locus that affects DNA repair GC strains FA1090 and its derivatives 1-81-S2 (uvrB:kan #4 Opa+) and 1-81-S2 (uvrB:kan #9) were tested. uvrB mutants have been used by other labs to reduce the amount of 8-MOP or S-59 needed to inactivate bacterial cells and to therefore leave them with higher metabolic activity (KBMA). [18, 20, 21] FA1090 and mutant strains were treated with varying doses of AMT, grown for one hour at 37º C, and then irradiated with UVA at 1 Joule/cm². The entire culture was washed and plated onto a single plate. Surviving colonies were counted and compared to live cultures to determine percent cell survival. Multiple cultures of each strain were tested at each dosage level. (see Table 3).

These low survival rates demonstrated that GC is extremely sensitive to the AMT/UVA treatment and there was no advantage in using a DNA repair mutant under these conditions. To get consistent inactivation of GC we treated FA1090 with two rounds of AMT and UVA. Thirty separate 1 ml cultures of FA1090 were given 20 μg/ml AMT and irradiated with 1 Joule/cm² UVA two times and plated onto 30 GC agar plates and grown for two days. No survivors were detected. This would indicate that out of approximately 3×10¹⁰ cells treated with AMT/UVA there were no cells still capable of growth.

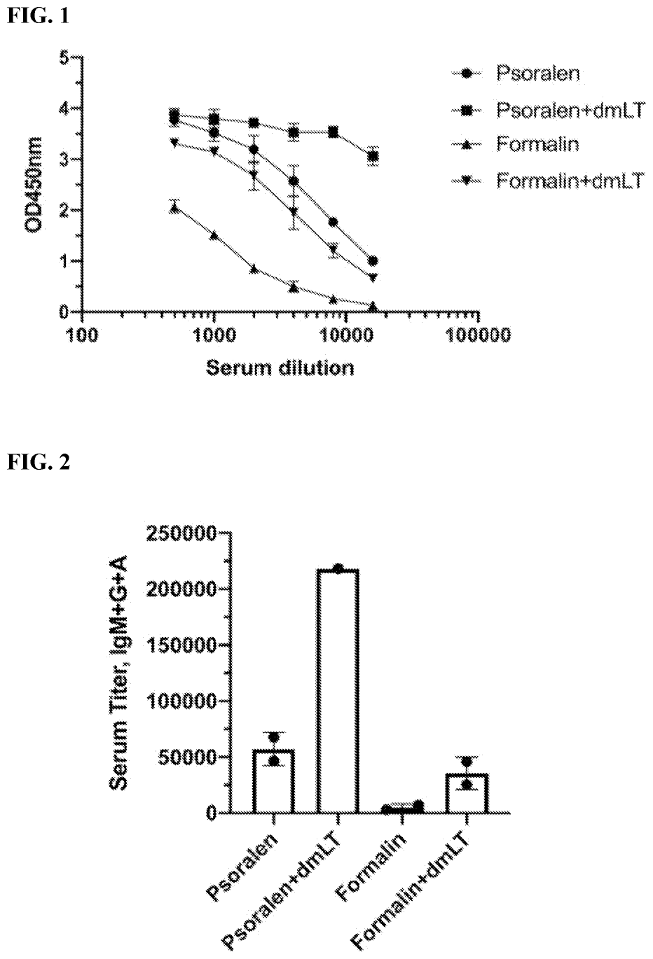

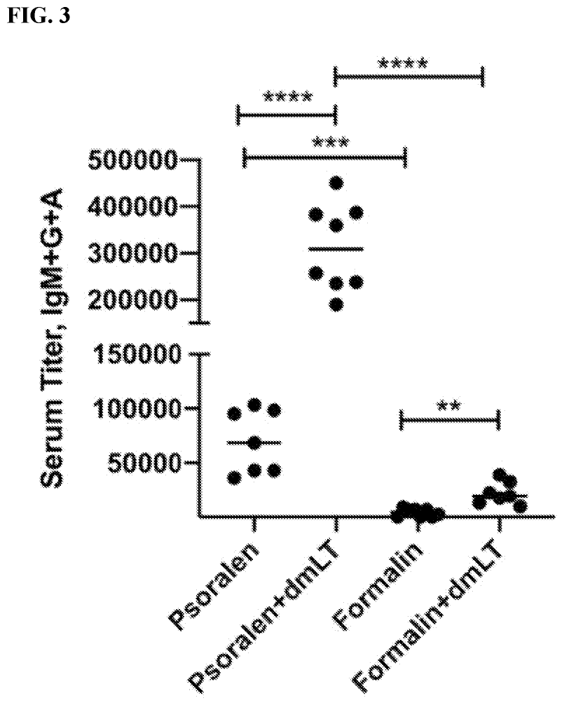

Vaccination of Mice: Psoralen-killed and formalin-killed whole cell vaccines were tested for immunogenicity in BALB/c mice. Vaccinations were done with and without dmLT adjuvant using a prime-boost regimen as described in materials and methods. Four groups of mice (7-8 mice/group) were primed by intramuscular injection of 5×10⁶ CFU equivalents of psoralen or formalin-inactivated bacteria in the presence or absence of dmLT (0.25 μg/mouse). The boost was performed with the same vaccines on day 21 and blood was collected on day 35 (14 after boost). Serum was analyzed for the presence of Neisseria gonorrhoeae-specific immunoglobulin (IgM+G+A) by whole cell ELISA using Neisseria gonorrhoeae as the coating antigen. At day 14 post-boost there was a demonstrable difference in serum antibody levels among the 4 groups of mice. The data in FIG. 1 depicts OD₄₅₀ values from pooled sera and FIG. 2 depicts titers from the pools, calculated based on serial dilutions of serum performed in 2 independent ELISA tests. FIG. 3 depicts antibody titers from individual mice and the geometric mean titer is indicated.

All mice produced a humoral response well above pre-immune levels (pre-immune titers of pooled sera from each group <500). However, as shown in FIG. 1, the psoralen-killed vaccines generated significantly greater antibody responses than did the formalin-killed vaccines, as shown by OD₄₅₀ vs dilution of pooled sera. Expressing the data as titers, shown in FIG. 2, Formalin-killed was approximately 5,000, and increased to approximately 35,000 in the presence of dmLT. The titer for the psoralen-killed vaccine was approximately 60,000 and was increased to approximately 200,000 in the presence of dmLT. The titers from individual mice are shown in FIG. 3. Significant differences in titer were demonstrated for psoralen vs formalin (p=0.0009), psoralen vs psoralen+dmLT (p<0.0001), formalin vs formalin+dmLT (p=0.0094) and psoralen\+dmLT vs formalin+dmLT (p<0.0001). These results indicate that as measured by the serum antibody response generated against a full repertoire of Neisseria gonorrhoeae antigens, the psoralen-killed vaccine was superior to the formalin-killed vaccine as generated by our methodology. In addition, the mucosal adjuvant dmLT enhanced the serum anti-Neisseria gonorrhoeae Ig response. Further studies will evaluate IgM and IgG (including subclasses of IgG) and mucosal (IgA) responses generated against the vaccines.

Development of a successful vaccine against Neisseria gonorrhoeae has been a challenge for decades. Only two gonorrhea vaccines, a partially autolyzed whole cell vaccine and a purified pilin vaccine, have been through human trials and neither were found to be effective. [24, 25] Although several target proteins are being investigated. [26, 27] no single antigen against which antibodies are protective has been identified. Therefore, an inactivated whole cell vaccine which could elicit an immune response against a broad variety of GC proteins is an appropriate platform for investigation. The challenge is finding a method to completely inactivate the cell without altering epitopes that are potentially important for generating protective immune responses.

A formalin inactivated whole cell GC vaccine administered to mice transdermally through microneedles was able to elicit an antibody response to formalin inactivated GC. This published method of inactivation was utilized for this study. Formalin inactivation is commonly utilized for whole cell vaccine development and could provide the diversity of antigenic presentation desired. However, the use of formalin as an inactivation agent has some drawbacks. Formalin treatment has been shown to cause intermolecular crosslinks between proteins that can cause conformational changes, which can lead to changed antigenic epitopes. [28-33]

Psoralen plus UVA inactivation, on the other hand, occurs at the nucleic acid level and therefore leaves the cell with its antigenic epitopes complete and unchanged. Psoralen is a furanocoumarin that intercalates with nucleic acids and upon exposure to long wave ultraviolet radiation leads to interstrand cross links by binding to pyrimidine bases. [34-36] Because psoralen plus UVA inhibits replication but does not affect protein structure or prevent translation, protein epitopes remain conserved and psoralen inactivated bacterial cells can be KBMA (killed but metabolically active) for at least a period of time. These properties of psoralen plus UVA inactivation could allow for the development of a more robust inactivated vaccine than other inactivation methods.

dmLT is an adjuvant derived from the heat labile enterotoxin (LT). It has been shown to lack the toxicity of its parent molecule and to promote immunity to codelivered antigens due to its immunostimulatory properties and to its universal cell binding. dmLT induces a variety of antigen specific immune responses including Th1, Th2, and Th17 and has also been shown to induce antigen specific IgA antibodies and long lasting memory when delivered mucosally. The purpose of the current study was to test the feasibility of psoralen inactivation as an approach to generate a psoralen-inactivated whole cell Neisseria gonorrhoeae vaccine and to evaluate immunogenicity in mice in direct comparison to formalin-inactivated bacteria in the presence and absence of the mucosal dmLT adjuvant.

The results showed a significant rise in antibody titers when mice were vaccinated with psoralen-inactivated whole cell GC when compared to formalin-inactivated whole cell GC. This supports our hypothesis that psoralen inactivation leaves more protein antigen epitopes intact than inactivating with formalin. Anti-GC antibody titers of mice vaccinated with psoralen inactivated GC were already higher after 21 days post prime and continued to be higher 2 weeks after the boost.

dmLT is a mucosal adjuvant. Gonorrhea is harbored in the urogenital tract, pharynx, and rectum, all sites that could benefit from an enhanced mucosal response. The results showed that dmLT raised antibody titers against whole cell GC to exceptionally high levels and made the mouse immunogenic response more uniform. The adaptive response to natural GC infection is poor. GC induces apoptosis in APCs and undergoes phase variation and antigenic variation that impedes bactericidal antibody production. [38, 39] The two cycles utilized for full activation may have allowed for more complete expression of varied antigens. In mice GC depresses the development of Th1 and Th2 adaptive immune responses and inhibits proliferation of T cells and B cells. [41, 42] It is possible that high antibody titers which are not present after natural infection but were seen with this vaccine and the addition of dmLT as an adjuvant could be effective in overcoming some of the obstacles against effective innate and adaptive responses.

This method of vaccine production for GC could easily be used against other organisms. No uvr mutants were needed for psoralen inactivation of GC, making the process easier. We have now been able to inactivate Shigella and Campylobacter jejuni (unpublished data) in a similar fashion. It is possible that psoralen plus UVA inactivated whole organism vaccines against other infections will prove to be more beneficial than the traditional formalin inactivated vaccines.

Accordingly, the present invention describes, among other features, a Neisseria gonorrhoeae bacterial vaccine and method of preparation thereof. Immunization with the described vaccine results in significant antibody responses against the pathogen's native unmodified proteins. The vaccine is prepared through inactivating Neisseria gonorrhoeae via exposure to psoralen followed by exposure to ultraviolet light (UVA). After the initial exposure to psoralen and UVA, a second application can be delivered to create a killed but metabolically active (KBMA) vaccine. This inactivation method completely inactivates the cell without altering epitopes that are potentially important for generating protective immune responses.

TABLE 1
Percentages of E. coli H10407 cultures inactivated by AMT
and UVA crosslinking. Multiple cultures were treated with different
doses of AMT and UV irradiation. The number of cultures that
were inactivated (had no overnight growth) is shown next to
the number of total cultures treated at the given levels of
AMT and UV light and is then expressed as a percentage.

| Dose of | | | |
|---|---|---|---|
| AMT (ug/ml) | 1 Joule/cm² | 7 Joules/cm² | 10 Joules/cm² |
| 0 | 0/6; 0% | 0/4; 0% | 0/2; 0% |
| 20 | 0/5; 0% | 2/3; 67% | |
| 30 | 0/5; 0% | 1/3; 33% | |
| 40 | 3/5; 60% | | |
| 50 | 9/11; 82% | 7/9; 78% | 12/15; 80% |
| 60 | 4/5; 80% | | |

TABLE 2
Per Cent Survival of H10407 treated with two doses of AMT and
two rounds of UV light irradiation at 1 Joule/cm². Multiple
cultures were treated with different doses of AMT and 1
Joule/cm² two times. A culture given no AMT was plated with
each experiment to determine total number of cells treated.

| Dose of | CFUs of | | |
|---|---|---|---|
| AMT (ug/ml) | treated cells | total cells treated | Percent Survival |
| 10 | 79 | 3 × 10⁹ | 2.6 × 10⁻⁶ |
| 30 | 2 | 4 × 10⁹ | 5 × 10⁻⁸ |
| 35 | 55 | 9 × 10⁹ | 6.1 × 10⁻⁷ |
| 40 | 0 | 3.7 × 10¹⁰ | 0 |

TABLE 3
Percent Cell Survival of FA1090 and uvr Mutant Strains when
treated with one Round of AMT and UV light Crosslinking. Multiple
cultures of FA1090 and of two of its uvrB mutants were treated with
different doses of AMT and 1 Joule/cm². A simultaneously grown
culture given no AMT was plated to determine an approximate
total number of cells treated in each trial.

| AMT dose | | | |
|---|---|---|---|
| (ug/ml) | FA1090 | uvrB: kan#4 | uvrB: kan#9 |
| 2 | 73/9.3 × 10⁸; | 2/3.6 × 10⁹ | 34/4.7 × 10⁹; |
| | 7.85 × 10⁻⁶ | 5.56 × 10⁻⁸ | 7.23 × 10⁻⁷ |
| 5 | 101/8.23 × 10⁹; | 0/2 × 10⁸; | 18/4.5 × 10⁹; |
| | 1.23 × 10⁻⁶ | 0 | 4.0 × 10⁻⁷ |
| 7 | 0/9.3 × 10⁸; | 0/2.4 × 10⁹; | 1/1.6 × 10⁹; |
| | 0 | 0 | 6.3 × 10⁻⁸ |
| 10 | 0/9.3 × 10⁸; | 0/2 × 10⁸; | 0/3.2 × 10⁸; |
| | 0 | 0 | 0 |
| 30 | 0/3.9 × 10⁸; | 0/4.5 × 10⁸; | 0/3.2 × 10⁸; |
| | 0 | 0 | 0 |
| 50 | 0/9.3 × 10⁸; | 0/4.5 × 10⁸; | 0/3.2 × 10⁸; |
| | 0 | 0 | 0 |
| 70 | 1/3.9 × 10⁸; | 5/4.5 × 10⁸; | 0/3.2 × 10⁸; |
| | 2.56 × 10⁻⁷ | 1.11 × 10⁻⁶ | 0 |
| 100 | 0/3.9 × 10⁸; | 0/4.5 × 10⁸; | 0/3.2 × 10⁸; |
| | 0 | 0 | 0 |

### References (All references listed below are incorporated herein by reference.)

1. Costa-Lourenço, A., Barros Dos Santos, K. T., Moreira, B. M., Fracalanzza, S., & Bonelli, R. R. (2017). Antimicrobial resistance in Neisseria gonorrhoeae: history, molecular mechanisms and epidemiological aspects of an emerging global threat. Brazilian journal of microbiology: [publication of the Brazilian Society for Microbiology], 48(4), 617-628.

2. World Health Organization; Geneva: 2016. WHO Guidelines for the Treatment of Neisseriagonorrhoeae. ncbi.nlm.nih.gov/books/NBK379221/.

3. Newman, L., et al., Global Estimates of the Prevalence and Incidence of Four Curable Sexually Transmitted Infections in 2012 Based on Systematic Review and Global Reporting. PLOS One, 2015. 10(12): p. e0143304.

4. Prevention, C.f.D.C.a., Antibiotic resistance threats in the United States, 2013 CDC Prevention-Threat Report, 2013. 2013: CDC, Atlanta, GA.

5. Workowski, K. A., Centers for Disease Control and Prevention Sexually Transmitted Diseases Treatment Guidelines. Clin Infect Dis, 2015. 61 Suppl 8: p. S759-62.

6. Farley, T. A., D. A. Cohen, and W. Elkins, Asymptomatic sexually transmitted diseases: the case for screening. Prev Med, 2003. 36(4): p. 502-9.

7. Liu, Y., B. Feinen, and M. W. Russell, New concepts in immunity to Neisseria gonorrhoeae: innate responses and suppression of adaptive immunity favor the pathogen, not the host. Front Microbiol, 2011. 2: p. 52.

8. Liu, Y., W. Liu, and M. W. Russell, Suppression of host adaptive immune responses by Neisseria gonorrhoeae: role of interleukin 10 and type 1 regulatory T cells. Mucosal Immunol, 2014. 7(1): p. 165-76.

9. Petousis-Harris, H., et al., Effectiveness of a group B outer membrane vesicle meningococcal vaccine against gonorrhoea in New Zealand: a retrospective case-control study. Lancet, 2017. 390(10102): p. 1603-1610.

10. Craig, A. P., et al., The potential impact of vaccination on the prevalence of gonorrhea. Vaccine, 2015. 33(36): p. 4520-4525.

11. Clements, J. D. and E. B. Norton, The Mucosal Vaccine Adjuvant LT(R192G/L211A) or dmLT. mSphere, 2018. 3(4).

12. LeCuyer, B. E., A. K. Criss, and H. S. Seifert, Genetic characterization of the nucleotide excision repair system of Neisseria gonorrhoeae. J Bacteriol, 2010. 192(3): p. 665-73.

13. Dillard, J. P., Genetic Manipulation of Neisseria gonorrhoeae. Curr Protoc Microbiol, 2011. Chapter 4: p. Unit4A 2.

14. Spence, J. M., L. Wright, and V. L. Clark, Laboratory maintenance of Neisseria gonorrhoeae. Curr Protoc Microbiol, 2008. Chapter 4: p. Unit 4A 1.

15. Gala, R. P., et al., Novel Whole-Cell Inactivated Neisseria Gonorrhoeae Microparticles as Vaccine Formulation in Microneedle-Based Transdermal Immunization. Vaccines (Basel), 2018. 6(3).

16. Jen, F. E., et al., The Neisseria gonorrhoeae Methionine Sulfoxide Reductase (MsrA/B) Is a Surface Exposed, Immunogenic, Vaccine Candidate. Front Immunol, 2019. 10: p. 137.

17. Schumacher, S. D. and J. Jose, Expression of active human P450 3A4 on the cell surface of Escherichia coli by Autodisplay. J Biotechnol, 2012. 161(2): p. 113-20.

18. Jalili, Z., et al., Characterization of killed but metabolically active uropathogenic Escherichia coli strain as possible vaccine candidate for urinary tract infection. Microb Pathog, 2018. 122: p. 184-190.

19. Brockstedt, D. G., et al., Killed but metabolically active microbes: a new vaccine paradigm for eliciting effector T-cell responses and protective immunity. Nat Med, 2005. 11(8): p. 853-60.

20. Lankowski, A. J. and E. L. Hohmann, Killed but metabolically active Salmonella typhimurium: application of a new technology to an old vector. J Infect Dis, 2007. 195(8): p. 1203-11.

21. Skoble, J., et al., Killed but metabolically active Bacillus anthracis vaccines induce broad and protective immunity against anthrax. Infect Immun, 2009. 77(4): p. 1649-63.

22. Maves, R. C., et al., Immunogenicity of a psoralen-inactivated dengue virus type 1 vaccine candidate in mice. Clin Vaccine Immunol, 2010. 17(2): p. 304-6.

23. Sundaram, A. K., et al., Comparison of purified psoralen-inactivated and formalin-inactivated dengue vaccines in mice and nonhuman primates. Vaccine, 2020. 38(17): p. 3313-3320.

24. Boslego, J. W., et al., Efficacy trial of a parenteral gonococcal pilus vaccine in men. Vaccine, 1991. 9(3): p. 154-62.

25. Greenberg, L., et al., Gonococcal vaccine studies in Inuvik. Can J Public Health, 1974. 65(1): p. 29-33.

26. Rice, P. A., et al., Neisseria gonorrhoeae: Drug Resistance, Mouse Models, and Vaccine Development. Annu Rev Microbiol, 2017. 71: p. 665-686.

27. Russell, M. W., A. E. Jerse, and S. D. Gray-Owen, Progress Toward a Gonococcal Vaccine: The Way Forward. Front Immunol, 2019. 10: p. 2417.

28. Carmichael, P.H.V.a.L.E., Dog response to inactivated canine parvovirus and feline panleukopenia virus vaccines. Cornell Vet, 1982. 72(1): p. 16-35.

29. Delrue, I., P. L. Delputte, and H. J. Nauwynck, Assessing the functionality of viral entry-associated domains of porcine reproductive and respiratory syndrome virus during inactivation procedures, a potential tool to optimize inactivated vaccines. Vet Res, 2009. 40(6): p. 62.

30. Fan, Y. C., et al., Formalin Inactivation of Japanese Encephalitis Virus Vaccine Alters the Antigenicity and Immunogenicity of a Neutralization Epitope in Envelope Protein Domain III. PLOS Negl Trop Dis, 2015. 9(10): p. e0004167.

31. Patil, P. K., et al., Integrity of GH-loop of foot-and-mouth disease virus during virus inactivation: detection by epitope specific antibodies. Vaccine, 2002. 20(7-8): p. 1163-8.

32. Rossio, J. L., et al., Inactivation of human immunodeficiency virus type 1 infectivity with preservation of conformational and functional integrity of virion surface proteins. J Virol, 1998. 72(10): p. 7992-8001.

33. Tano, Y., et al., Antigenic characterization of a formalin-inactivated poliovirus vaccine derived from live-attenuated Sabin strains. Vaccine, 2007. 25(41): p. 7041-6.

34. Hanson, C. V., Photochemical inactivation of viruses with psoralens: an overview. Blood Cells, 1992. 18(1): p. 7-25.

35. Hanson, C. V., J. L. Riggs, and E. H. Lennette, Photochemical inactivation of DNA and RNA viruses by psoralen derivatives. J Gen Virol, 1978. 40(2): p. 345-58.

36. Wollowitz, S., Fundamentals of the psoralen-based Helinx technology for inactivation of infectious pathogens and leukocytes in platelets and plasma. Semin Hematol, 2001. 38(4 Suppl 11): p. 4-11.

37. Duncan, J. A., et al., Neisseria gonorrhoeae activates the proteinase cathepsin B to mediate the signaling activities of the NLRP3 and ASC-containing inflammasome. J Immunol, 2009. 182(10): p. 6460-9.

38. Rotman, E. and H. S. Seifert, The genetics of Neisseria species. Annu Rev Genet, 2014. 48: p. 405-31.

39. Stern, A., et al., Opacity genes in Neisseria gonorrhoeae: control of phase and antigenic variation. Cell, 1986. 47(1): p. 61-71.

40. Liu, Y., et al., Neisseria gonorrhoeae selectively suppresses the development of Th1 and Th2 cells, and enhances Th17 cell responses, through TGF-beta-dependent mechanisms. Mucosal Immunol, 2012. 5(3): p. 320-31.

41. Pantelic, M., et al., Neisseria gonorrhoeae kills carcinoembryonic antigen-related cellular adhesion molecule 1 (CD66a)-expressing human B cells and inhibits antibody production. Infect Immun, 2005. 73(7): p. 4171-9.

42. Zhu, W., et al., Neisseria gonorrhoeae suppresses dendritic cell-induced, antigen-dependent CD4 T cell proliferation. PLOS One, 2012. 7(7): p. e41260.

The foregoing is illustrative of the present invention, and is not to be construed as limiting thereof. The invention is defined by the following claims, with equivalents of the claims to be included therein.

1. An immunogenic composition comprising a crosslinked inactivated bacterium, wherein the bacterium has been crosslinked and inactivated by contact with psoralen or a psoralen derivative and exposure to ultraviolet (UV) irradiation.

2. The immunogenic composition of claim 1, wherein the bacterium is a gram-negative bacterium.

3. The immunogenic composition of claim 2, wherein the bacterium is Neisseria gonorrhoeae, Escherichia coli, Shigella, and/or Campylobacter jejuni.

4. The immunogenic composition of claim 1, wherein said psoralen or psoralen derivative is selected from the group consisting of 8-Methoxypsoralen (8-MOP), 4,5′,8-Trimethylpsoralen (TMP), psoralen derivative 4-aminomethyl-4,5′, 8-trimethylpsoralen (AMT) and a combination thereof.

5. The immunogenic composition of claim 1, wherein the UV irradiation comprises UVA irradiation.

6. The immunogenic composition of claim 1, comprising the inactivated crosslinked bacterium in a concentration of about 5×10⁶ CFU equivalents to about 5×10⁸ CFU equivalents.

7. (canceled)

8. The immunogenic composition of claim 1, further comprising an adjuvant and/or a pharmaceutically acceptable carrier.

9. (canceled)

10. The immunogenic composition of claim 9, wherein the composition comprises the adjuvant at a concentration of about 0.25 μg/ml to about 250 μg/ml.

11-16. (canceled)

17. A method of making an immunogenic composition comprising:
a) contacting a live bacterium to psoralen or psoralen derivative in a medium to produce a bacterium/psoralen mixture, and
b) exposing the mixture of step (a) to an intensity of ultraviolet (UV) radiation for a time period sufficiently long to crosslink the live bacterium and render the live bacterium inactive.

18. (canceled)

19. The method of claim 17, wherein the step (b) exposing the mixture of step (a) to an intensity of ultraviolet (UV) radiation for a time period sufficiently long to render the live bacterium inactive comprises repeating steps (a) and/or (b) iteratively until complete inactivation.

20. The method of claim 19, wherein the steps (a) and/or (b) are repeated for one or more iterations, and wherein the mixture is incubated for a time period between iterations of additional step (a) contacting with psoralen or a psoralen derivative and/or step (b) exposing the mixture of step (a) to the ultraviolet (UV) radiation.

21. (canceled)

22. The method of claim 17, wherein said psoralen or psoralen derivative is selected from the group consisting of, 8-Methoxypsoralen (8-MOP), 4,5′,8-Trimethylpsoralen (TMP), psoralen derivative 4-aminomethyl-4,5′, 8-trimethylpsoralen (AMT) and a combination thereof.

23. The method of claim 17, wherein said psoralen is added to the medium at a concentration of about 0.01 to about 600 μg/ml, wherein the UV radiation exposure is about 2 to about 30 minutes, and/or wherein the intensity of UV radiation is about 0.01 Joule/cm² to about 100 Joule/cm².

24-28. (canceled)

29. An immunogenic composition produced by the method of claim 17.

30. A method of producing an immune response to a bacterium in a subject, comprising administering to the subject an effective amount of the immunogenic composition of claim 1, thereby producing an immune response to a bacterium in the subject, wherein the subject is a mammal.

31. A method of treating a bacterial infection in a subject in need thereof, comprising administering to the subject an effective amount of the immunogenic composition of claim 1, thereby treating a bacterial infection in the subject, wherein the subject is a mammal.

32. A method of preventing or reducing the risk of a disease or disorder associated with or caused by a bacterial infection in a subject, comprising administering to the subject an effective amount of the immunogenic composition of claim 1, thereby preventing or reducing the risk of a disease or disorder associated with or caused by a bacterial infection in the subject, wherein the subject is a mammal.

33. A method of protecting a subject from the effects of a bacterial infection, comprising administering to the subject an effective amount of the immunogenic composition of claim 1, thereby protecting the subject from the effects of a bacterial infection, wherein the subject is a mammal.

34-35. (canceled)

36. A method of preventing or reducing the risk of a gonococcal infection or disorder associated with or caused by a gonococcal infection in a subject, comprising administering to the subject an effective amount of the immunogenic composition of claim 1, thereby preventing or reducing the risk of a disease or disorder associated with or caused by a bacterial infection in the subject, wherein the subject is a mammal.

37. A method of protecting a subject from the effects of a gonococcal infection, comprising administering to the subject an effective amount of the immunogenic composition of claim 1, thereby protecting the subject from the effects of a bacterial infection, wherein the subject is a mammal.

38-46. (canceled)
